(19) United States
(12) Patent Application Publication
(10) Pub. No.: US 2007/0031854 A1
(43) Pub. Date: Feb. 8, 2007

(54) Novel 10,10'-substituted-9,9'-biacridilidine luminescent molecules their preparation and uses

(76) Inventor: George William Katsilometes, Pocatello, ID (US)

Correspondence Address: George William Katsilometes, D.V.M., Ph.D., 1424 East Maple Street, Pocatello, ID, 83201, US

(21) Appl. No.: 11/263,117

(22) Filed: Nov. 1, 2005

Related U.S. Application Data
(63) Continuation-in-part of application No. 10/388,733, filed on Mar. 14, 2003, now abandoned.
Application No. 10/388,733 is a continuation-in-part of application No. 09/241,513, filed on Feb. 1, 1999, now abandoned.

(51) Int. Cl. C12Q 1/68; G01N 33/53; C07K 14/47; C07K 16/46; C07D 401/14

(52) U.S. Cl. 435/6; 435/7.1; 530/409; 536/25.32; 546/102; 530/391.1

(57) ABSTRACT

Novel symmetric, uniformly symmetric and asymmetric 10,10′-substituted-9,9′-biacridines and the synthesis of such symmetric, uniformly symmetric and asymmetric 10,10′-substituted-9,9′-biacridine molecules and their derivatives is disclosed. These molecules are shown to produce light by luminescence in the presence of signals. These symmetric, uniformly symmetric or asymmetric 10,10′-substituted-9,9′-biacridines are used alone or attached to haptens or macromolecules and are utilized as labels in the preparation of iluminescent homogeneous and heterogeneous assays. They are also used in conjunction with other label molecules to produce multiple analyte assays.

### APPLICATION HISTORY AND RELATED APPLICATIONS

This application is a continuation-in-part application of U.S. patent application Ser. No. 10/388,733, filed on Mar. 14, 2003, U.S. patent application Ser. No. 09/241,513, filed on Sep. 24, 2001, which is in turn a continuation of U.S. patent application Ser. No. 09/241,513 filed on Feb. 1, 1999.

### BACKGROUND OF THE INVENTION

1. Field of the Invention

The present invention relates to luminescence, to luminescent systems, and to novel compounds used in luminescent systems and their methods of synthesis.

2. Background of the Art

Luminescence is the emission of light without significant amounts of attendant heat. A specific light-emitting state is effected within a chemical without greatly increasing the temperature of the chemical. The light-emitting state is usually effected by providing energy to a molecule, thereby generating the light-emitting state. The particular wavelength of radiation, which is emitted by the molecule, is determined by the characteristics of the molecule, and that wavelength does not usually change when the energy source to the molecule is changed.

Chemiluminescence is a special situation of luminescence, where the energy is directed to the molecule by physical or chemical reaction. Chemiluminescence involves substantially direct conversion of physical or chemical energy to light energy. A number of different classes of compounds have been found to be particularly susceptible to chemiluminescent reactions. Exemplary classes of chemiluminescent compounds are phthalazinediones, especially the 2,3-dihydro-1,4-phthalazinediones (e.g., EPO 0 116 454 A2, T. H. Whitehead et al., 1985; and U.S. Pat. No. 4,853,327, N. Dattagupta).

implement this type of method (Schroeder et al., Methods in Enzymology, 17:24-462 (1978); Zeigler, M. M., and T. O. Baldwin, Current Topics In Bioenergetics, D. Rao Sanadi ed. (Academic Press) pp. 65-113 (1981); DeLuca, M., Non-Radiometric Assays: Technology and Application in Polypeptide and Steroid Hormone Detection, (Alan R. Liss, Inc.) pp. 47-60 and 61-77 (1988); DeJong, G. J., and P. J. M. Kwakman, J. of Chromatography, 492:319-343 (1989); McCapra, F. et al., J. Biolumin. Chemilumin., 4:51-58 (1989); Diamandis, E. P., Clin. Biochem., 23:437-443 (1990); Gillevet, P. M., Nature, 348:657-658 (1990); Kricka, L. J., Amer. Clin. Lab., Nov/Dec:30-32 (1990)).

Chemiluminescence is the emission of light by means of physical or chemical reaction and may be further defined as the emission of light during a reversion to the ground state of electronically excited products of these reactions ((Woodhead, J. S. et al., Complementary Immunoassays, W. P. Collins ed. (John Wiley & Sons Ltd.), pp. 181-191 (1988)). Chemiluminescent reactions are often discussed in terms of either enzyme-mediated or nonenzymatic reactions. It has been known for some time that the luminescent reactant luminol can be oxidized in neutral to alkaline conditions (pH 7.0-10.2) in the presence of: a) oxidoreductase enzymes (horseradish peroxidase, xanthine oxidase, glucose oxidase), b) H₂O₂, c) certain inorganic metal ion catalysts or molecules (iron, manganese, copper, zinc), and d) chelating agents, and that this oxidation leads to the production of an excited intermediate (3-aminophthalic acid) which emits light on decay to its ground state ((Schroeder, H. R. et al., Anal. Chem., 48:1933-1937 (1976); Simpson, J. S. A. et al., Nature, 279:646-647 (1979); Baret, A., U.S. Pat. No. 4,933,276)). Other specific molecules and derivatives used to produce luminescence include cyclic diacyl hydrazides other than luminol (e.g., isoluminols), dioxetane derivatives, acridinium derivatives and peroxyoxylates ((Messeri, G. et al., J. Biolum. Chemilum., 4:154-158 (1989); Schaap, A. P. et al., Tetrahedron Lett., 28:935-938 (1987); Givens, R. S. et al., ACS Symposium Series 383; Luminescence Applications, M. C. Goldberg ed. (Amer. Chem. Soc., Wash. D.C., pp. 127-154 (1989)). Additional molecules which produce light and have been utilized in the ultra sensitive measurement of molecules are polycyclic and reduced nitropolycyclic aromatic hydrocarbons, polycyclic aromatic amines, fluoresamine-labeled catecholamines, and other fluorescent derivatizing agents such as the coumarins, ninhydrins, o-phthalaldehydes, 7-fluoro-4-nitrobenz-2,1,3-oxadiazoles, naphthalene-2,3-dicarboxaldehydes, cyanobenzisoindoles and dansyl chlorides ((Simons, S. S., Jr. and D. F. Johnson, J. Am. Chem. Soc., 98:7098-7099 (1976); Roth, M., Anal. Chem., 43:880-882 (1971); Dunges, W. ibid, 49:442-445 (1977); Hill, D. W. et al., ibid, 51:1338-1341 (1979); Lindroth, P. and K. Mopper, ibid, 51:1667-1674 (1979); Sigvardson, K. W. and J. W. Birks, ibid, 55:432-435 (1983); Sigvardson, K. W. et al., ibid, 56:1096-1102 (1984); de Montigny, P. et al., ibid, 59:1096-1101 (1987); Grayeski, M. L. and J. K. DeVasto, ibid, 59:1203-1206 (1987); Rubinstein, M. et al., Anal. Biochem., 95:117-121 (1979); Kobayashi, S.-I. et al., ibid, 112:99-104 (1981); Watanabe, Y. and K. Imai, ibid, 116:471-472 (1981); Tsuchiya, H., J. Chromatog., 231:247-254 (1982); DeJong, C. et al., ibid, 241:345-359 (1982); Miyaguchi, K. et al., ibid, 303:173-176 (1984); Sigvardson, K. W. and J. W. Birks, ibid, 316:507-518 (1984); Benson, J. R. and P. E. Hare, Proc. Nat. Acad. Sci., 72:619-622 (1975); Kawasaki, T. et al., Biomed. Chromatog., 4:113-118 (1990)).

There are currently seven known nonenzymatic systems which can be utilized as labels in testing: 1) the acridinium derivatives ((McCapra et al., British Patent No. 1,461,877; Wolf-Rogers J. et al., J. Immunol. Methods, 133:191-198 (1990)); 2) isoluminols, 3) metalloporphyrins (Forgione et al., U.S. Pat. No. 4,375,972); 4) nonmetallic tetrapyrroles (Katsilometes, U.S. Pat. No. 5,340,714); 5) electrochemiluminescence (Fiaccabrino, G. C. et al, Anal. Chim. Acta, 359(3): 263-267 (1998); 6) the derivatized biacridines (Katsilometes, et al., U.S. Pat. No. 5,866,335). and 7) the singlet oxygen responders ((McCapra, F and Hann, R. A., J. Chem. Soc., Chem. Commun.,: 442-443 (1969); Ullman et al. U.S. Pat. No. 6,143,514; Katsilometes, U.S. patent application Ser. No. 10/388,733)). These seven non-enzymatic systems have certain advantages over the enzyme-mediated systems in that they tend to have faster kinetics resulting in peak light output within seconds. The metalloporphyrins are small hapten molecules, which decrease steric hindrance problems in antigen binding. In addition, the metalloporphyrin molecules known to be luminescent are those containing a paramagnetic metal ion with emission yields above 10⁻⁴ ((Gouterman, M., The Porphyrins, Vol. III, Dolphin, D., ed. (Academic Press): 48-50, 78-87, 115-117, 154-155 (1978); Canters, G. W. and J. H. Van Der Waals, ibid, 577-578)). It is known that metalloporphyrins, hyposporphyrins, pseudonormal metalloporphyrins and metalloporphyrin-like molecules such as metallic chlorins, hemes, cytochromes, chlorophylls, lanthanides and actinides undergo oxidation/reduction reactions which are either primary or secondary to structural perturbations occurring in the metallic center of these molecules and that their reactive ability to catalyze the production of chemiluminescence has been ascribed to the metallo center of these molecules ((Eastwood, D. and M. Gouterman, J. Mol. Spectros., 35:359-375 (1970); Fleischer, E. B. and M. Krishnamurthy, Annals N.Y. Academy of Sci., 206:32-47 (1973); Dolphin, D. et al., ibid, 206:177-201; Tsutsui, M. and T. S. Srivastava, ibid, 206:404-408; Kadish, K. M. and D. G. Davis, ibid, 206:495-504; Felton, R. H. et al., ibid, 206:504-516; Whitten, D. G. et al., ibid, 206:516-533; Wasser, P. K. W. and J. -H. Fuhrhop, ibid, 206:533-549; Forgione et al., U.S. Pat. No. 4,375,972; Reszka, K. and R. C. Sealy, Photochemistry and Photobiology 39:293-299 (1984); Gonsalves, A. M. d'A. R. et al., Tetrahedron Lett., 32:1355-1358 (1991)). These reactions are altered by iron and other metal ions which may be present in the reactants, and these metal ions can interfere with and greatly confound the assay of metalloporphyrin conjugate concentrations ((Ewetz, L. and A. Thore, Anal. Biochem., 71:564-570 (1976)). Different metals will strongly influence the lifetimes and luminescent properties of the metalloporphyrins.

The nonmetallic porphyrin deuteroporphyrin-IX HCl has been shown to luminesce and to mediate the production of light from luminol in solution (Katsilometes, G. W., supra).

Use of the luminescent acridinium ester and amide derivatives in chemiluminescent reactions and in the development of nonisotopic ligand binding assays has been reported and reviewed ((Weeks, I. et al., Clin. Chem., 29/8:1474-1479 (1983); Weeks, I. and J. S. Woodhead, Trends in Anal. Chem. 7/2:55-58 (1988)). The very short lived emission of photons (<5 sec) to produce the flash-type kinetics in the presence of H₂O₂ and NaOH oxidation reagents (pH 13.0) is characteristic of the system.

Methods of preparation of acridones and variously substituted acridines and acridones have been summarized ((Acridines, Acheson, R. M. and L. E. Orgel (Interscience Publishers, N.Y.) pp. 8-33, 60-67, 76-95, 105-123, 148-173, 188-199, 224-233 (1956)). Formation of biacridines by the combining of two acridine residues at the carbon-9 atom has been described and reviewed previously ((Gleu, K. and R. Schaarschmidt, Berichte, 8:909-915 (1940)). These efforts led to the synthesis of 10,10′-dimethyl, 10,10′-diphenyl and 10,10′-diethyl-9,9′-biacridinium nitrate molecules. It was also reported that these molecules will produce light when exposed to hydrogen peroxide in basic solution ((Gleu, K. and W. Petsch, Angew. Chem., 48:57-59 (1935); Gleu, K. and R. Schaarschmidt, Berichte, 8:909-915 (1940)).

The mechanism of light production by lucigenin (10,10′-dimethyl-9,9′-biacridinium nitrate) has been extensively studied and has been ascribed to a series of hydroxide ion nucleophilic additions to acridinium salts and their reduction products (pinacols), culminated by the oxidation of the main end product N-methylacridone ((Janzen, E. G. et al., J. Organic Chem., 35:88-95 (1970); Maeda, K. et al., Bul. Chem. Soc. Japan, 50:473-481 (1977); Maskiewicz, R. et al., J. Am. Chem. Soc., 101/18:5347-5354 (1979); Maskiewicz, R. et al., ibid, 101/18:5355-5364 (1979)). It has also been shown that lucigenin can produce light following divalent reduction to 10,10′-Dimethyl-9,9′-biacridylidine which can be attacked by oxygenating species, such as singlet oxygen to yield the moloxide (dioxetane) intermediate which disintegrates yielding one excited and one ground state N-methylacridone ((McCapra, F and Hann, R. A., J. Chem. Soc., Chem. Commun.,: 442-443 (1969); Ullman et al. U.S. Pat. No. 6,143,514)).

Modifications and derivatizations of 10-methyl acridine at the carbon-9 atom have led to the production of several useful chemiluminescent molecules having varying degrees of stability (Law, S.-J. et al., J. Biolum. Chemilum., 4:88-98 (1989)). These molecules produce a flash of light lasting less than five seconds when exposed to 0.5% w/v hydrogen peroxide in 0.1 mol/L nitric acid followed by a separate solution containing 0.25 mol/L sodium hydroxide.

A luminescent derivative, a luminescent derivatized molecule or a derivatized luminescent molecule as defined herein is a molecule which results from the covalent binding of a functional group or a group which changes the chemical reactivity and properties of a precursor molecule leading to the formation of a luminescent molecule suitable for conjugation to an analyte or a particular binding partner one wishes to use in assays. A N-hydroxy succinimide derivatization of biacridines at one or both of the two 10,10′ positions are the preferred luminescent derivatives of the invention. A compound or a molecule is a “derivative” of a first compound or first molecule if the derivative compound or molecule is formed (or can be formed) by reaction of the first compound or first molecule to form a new compound or new molecule either smaller or larger than the first compound or first molecule while retaining at least part of the structure of the first compound or first molecule. As used herein the term “derivative” can also include a “luminescent derivative”.

Prior to the invention described in U.S. patent application Ser. No. 08/265,481 (filed Jun. 24, 1994), PCT Application WO 96/00392 (published Apr. 1, 1996), and Ser. No. 08/767,288 (filed Dec. 16, 1996), synthesis of derivatized luminescent 10,10′-substituted-9,9′-biacridines had not been achieved. The previously known luminescent biacridines (e.g., lucigenin) contained no reactive group(s), which permitted the conjugation of the biacridine molecule to another. Until then, the biacridines had academic interest only and were used to study the mechanism of light production and the interactions of reactive ionic species.

The use of luminescent reactions at the surface of light conductive materials (e.g., fiber-optic bundle) is the basis of the development of luminescent sensors or probes (Blum, L. J. et al., Anal. Lett., 21:717-726 (1988)). This luminescence may be modulated by specific protein binding (antibody) and can be produced in a micro environment at the surface of the probe. The light output is then measured by photon measuring devices in the formulation of homogeneous (separation free) assays (Messeri, G. et al., Clin. Chem., 30:653-657 (1984); Sutherland, R. M. et al., Complementary Immunoassays, Collins, W. P., ed. (John Wiley & Sons, Ltd.) pp. 241-261 1988)).

It is possible to improve assay sensitivity by improving the efficiency of light output obtained from luminescent reactions. This has been accomplished by synthesizing derivatized biacridine molecules capable of producing superior quantities of photon emissions and/or by improving existing signal conditions producing a greater intensity of light during luminescent reactions. The ability to modulate the kinetics and wavelengths of light output through manipulation of the biacridine structure and signal is particularly beneficial in tailoring assays for a variety of uses (genetic probe, sensor, macromolecular assays, hapten assays, particle assays, high throughput screening, and the like).

### SUMMARY OF THE INVENTION

The present invention relates to novel 10,10′-substituted-9,9′-biacridilidine and 10,10′-substituted-9,9′-biacridine epoxide compounds and the synthesis of these new biacridilidine and biacridine epoxide compounds, the production of light from these new molecules, and the use of these new molecules in luminescent reactions and assays. More particularly, the invention describes the synthesis of symmetrical, uniformly symmetrical and asymmetrical 10,10′-para-substituted-9,9′-biacridilidenes and biacridine epoxides, particularly 10-para-toluic acid (and derivatives, such as ester derivatives), 10′-para-substituted-9,9′-biacridilidenes and biacridine epoxides and the demonstration of the ability to covalently bind (conjugate) these new molecules to other molecules such as antibody, hapten, streptavidin, biotin, nucleic acid or any other appropriate substance for which detection and/or quantification is desired and to produce measurable light from biacridilidene and biacridine epoxide luminescent label molecules. The invention further involves luminescent generating systems comprising or producing at least one oxidant, ozone, singlet oxygen, phosphite, halogen, hydrogen peroxide, superoxide, radiofrequency discharge, microwave, ultrasound, electromagnetic energy, heat, energy or light to produce high yield photon emissions useful in assays, physical assays, chemical assays, nucleic acid assays and immunoassays.

The terminology used in the practice of the present invention with regard to symmetry, uniform symmetry and asymmetry on these 10,10′-para-substituted-9,9′-biacridilidene and biacridine oxide compounds has specific meaning as used in the practice of the present invention, that is not inconsistent with conventional use of the terms. The terms “symmetric” and “symmetry” as used in the present invention mean that the actual groups on the 10-position and the 10′-position of the compounds are identical, irrespective of any other substitution on the 10,10′-para-substituted-9,9′-compounds. That is, if the 10-position and the 10′-position are substituted with the identical group (e.g., p-toluic acid), the compounds are within the definition of symmetric. If there are identical substituents on the 10-position and the 10′-position of the compound and the remainder of the positions on the acridinium moieties have identical substitution thereon, the compounds would be referred to as “uniformly symmetric” in the practice of the present invention. Thus, a compound such as 10,10′-para-toluic acid-9,9′-biacridilidene is uniformly symmetric, but a compound 10,10′-para-toluic acid-2-fluoro-9,9′-biacridilidene is within the definition of symmetric (as would be the 10,10′-para-toluic acid-9,9′-biacridilidene) because both the 10 and the 10′ positions have the same substituent. A compound such as 10-para-toluic acid-10′-methyl-biacridilidene would not be symmetric and would be classified as asymmetric according to the present invention. It is also to be noted that throughout this text the term biacridine is used. Biacridene is a generic term encompassing all biacridiniums, biacridinium salts, biacridilidenes, biacridene oxides (epoxides) and any other molecules having Bis-acridone nuclei. The terms biacridilidene and biacridene epoxide are used. The term “biacridilidene” is used to denote the species having a double bond between the 9 and 9′ positions while the term “biacridene epoxide (oxide)” denotes the species having an interposed oxygen atom between the 9 and 9′ positions. This use of nomenclature is solely for purposes of convenience and is not an attempt to differentiate the invention between these compounds.

The classification of compounds within the terminology of symmetric uniformly symmetric or asymmetric according to the present invention is not affected by restricted rotation giving rise to perpendicular disymmetric planes, as where the compounds have chiral centers and form mirror image orientations. Even though these structures can be graphically or schematically represented such that no possible perpendicular planes can be drawn which can be bisected by a plane as in visual symmetry, the term symmetry as used herein applies to chemical substitution only, not to mere physical orientation or mere optical activity and orientation.

One aspect of the invention is the method for detecting the presence of the novel biacridilidene and biacridine epoxide luminescent compounds in a sample. The method comprises contacting the sample with signal to produce, by means of physical or chemical luminescence, measurable emitted light and measuring the emitted light with a photometric sensor, instrument or device.

Another aspect of this invention is methods for the synthesis of luminescent symmetric, uniformly symmetrical and asymmetric 10, 10′-substituted-9,9′-biacridilidene and biacridine epoxide molecules, which can be bound to analyte or to binding partner of analyte or to ligand of binding partner to analyte. These molecules may have additional substitutions at other sites on the molecule such as carbon atoms 1 through 8 on the acridone precursor by selection of the appropriate reagent (e.g., the appropriately substituted acridone precursor). Preferred example substituents on the 1 through 8 carbon atoms of the biacridilidene and biacridine epoxide compounds may be selected from the group consisting of alkyl, alkenyl, aryl, amino, substituted amino, carboxyl or hydroxyl groups (i.e., substituted or not), with halogenated alkyl (especially perfluorinated alkyl groups) particularly contemplated, halogen (fluorine, bromine, chlorine and iodine), sulfonate, alkoxy, aryloxy, carboxyl, nitrile, inorganic acids groups (e.g., sulfonic acid, phosphonic acid), hetero atoms as bridging groups (e.g., sulfur, nitrogen, oxygen, boron), and the like.

Still another aspect of the invention is directed to a luminescent system for emitting measurable light useful in a physical assay, chemical assay, a ligand binding assay, an immunoassay or a nucleotide assay. The system comprises uniformly symmetrical, symmetrical or asymmetrical 10,10′-substituted-9,9′-biacridilidenes and biacridine epoxides with specific energy of activation and oxidation potential, bound to analyte, or to binding partner of analyte or to ligand of binding partner to analyte and signal energy, electromagnetic energy, radiofrequency discharge, ultrasound, microwave, heat, light, singlet oxygen, oxidant or combination of signals capable of overcoming the inherent energy of activation of biacridilidene and biacridine epoxide. In this system the biacridilidene and biacridine epoxide act as a luminescent label (tag) for the production of luminescence in physical assays, chemical assays, homogeneous, heterogeneous competitive and sandwich immunoassays, ligand binding assays and nucleotide assays. The light is produced upon exposure of the biacridilidene label to signal overcoming the inherent energy of activation of biacridilidene and biacridine epoxide.

The asymmetrical, symmetrical and uniformly symmetrical 10,10′-substituted-9,9′-biacridilidene and biacridine oxide are especially beneficial as the labels are more sensitive (i.e., detect smaller quantities of analyte) than known luminescent labels and are able to undergo modification of the kinetics and wavelength of light production though manipulation of the structure and/or signal environment (e.g. by exposure to electromagnetic energy, oxygen, hydrazides, fluorophores and/or photosensitizers) resulting in light production for at least 0.0001 second and also for as long as 6 seconds or longer.

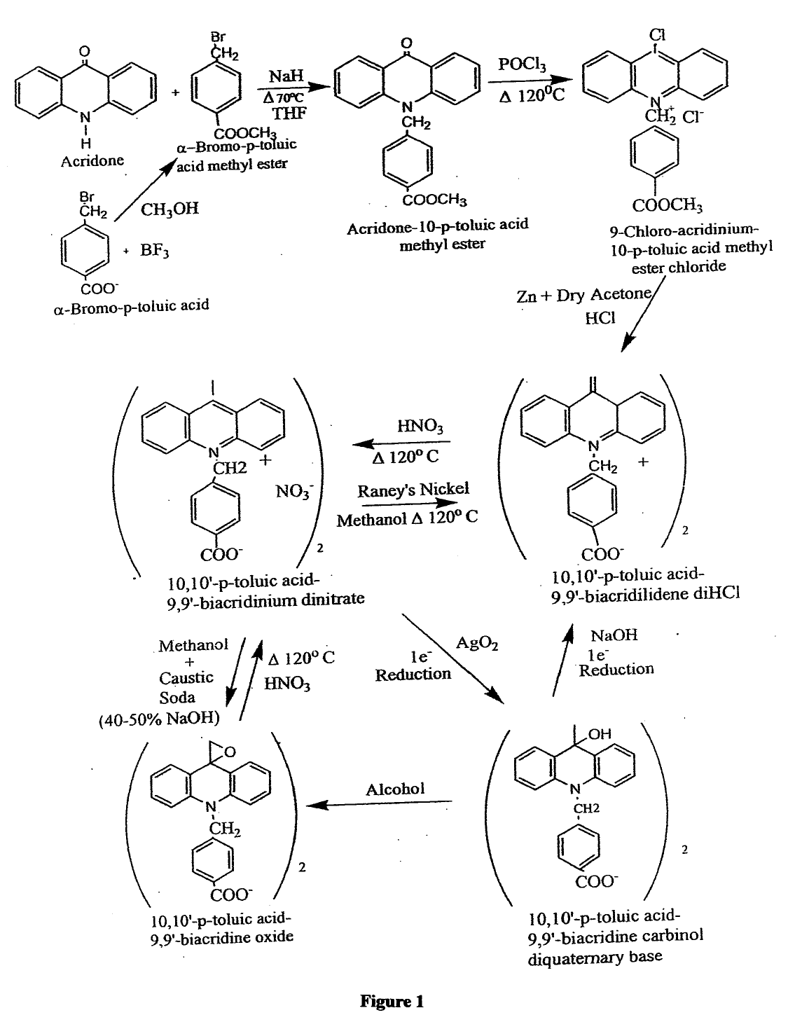

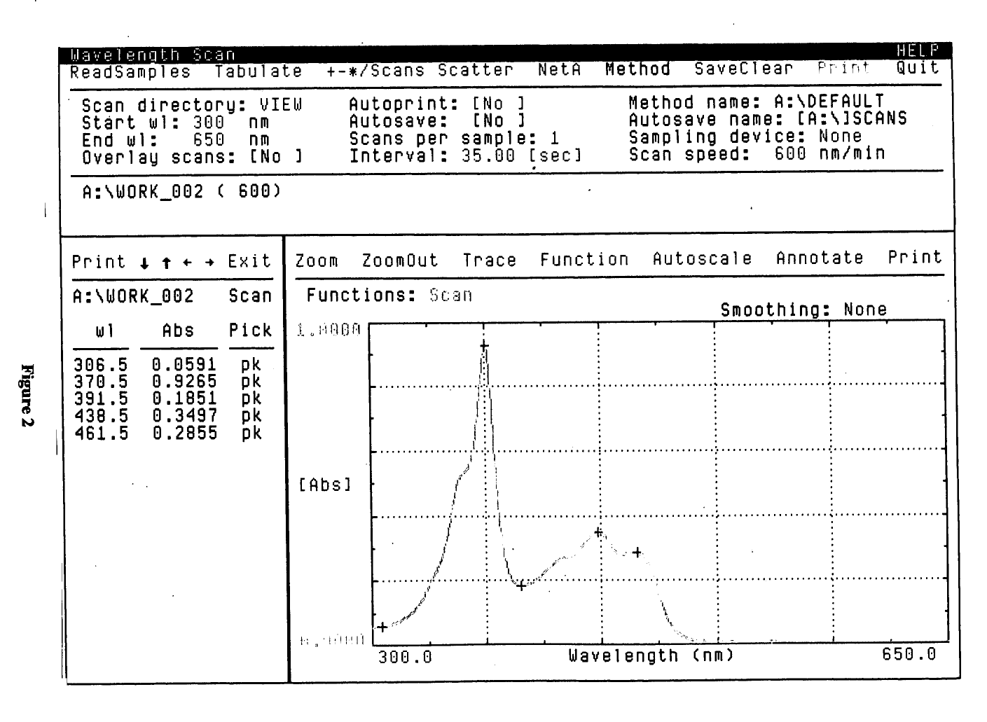

A further aspect of the invention is a method for the synthesis of novel asymmetrical, uniformly symmetrical and symmetrical 10,10′-substituted-9,9′-biacridilidenes and biacridine epoxides, which have luminescent properties. These methods involve, for example, the alkylation of the starting material (acridone or selectively substituted acridone) to form the corresponding N-substituted acridone followed by dimerization of this product. Oxidation of the resulting acrydilidine is accomplished with an oxidizing acidic environment, preferably with an oxidizing acid, such as nitric acid, to yield the 10,10′-substituted-9,9′-biacridinium di(anion), e.g., dinitrate. If necessary, the biacridilidene may be derivatized by reaction with, for example, N-hydroxysuccinimide and dicyclohexylcarbodiimide to give the corresponding N-hydroxysuccinimide (NHS) ester. The resulting biacridine can be divalently reduced to give the biacridilidene. The epoxide can be synthesized by exposure of the biacridinium salt to a methanolic solution of caustic soda or by exposure of the biacridine carbinol diquaternary base (formed by AgO₂ reduction of the biacridinium salt) to alcohol (FIG. 1 and General Formula (1c)).

Another aspect of the invention is luminescent signal which when reacted with luminescent label that is luminescent molecule produces light. The signals comprise, for example, singlet oxygen, electromagnetic energy, energy, radiofrequency discharge, ultrasound, microwave, heat, electricity, light, oxidant or combination of signals capable of overcoming the inherent energy of activation of biacridilidene. Where the luminescent label is biacridilidene derivative, biacridine epoxide derivative, complexes of biacridilidene, complexes of biacridine epoxide the reaction will produce a signal-to-noise photon emission ratio of at least 20:1 at 1 ng/ml of label concentration for at least 0.0001 second duration. Depending upon the label and the variation of the concentration of the signal, the signal-to-noise ratio can be 50:1, 100:1, 200:1, 500:1, and even 700:1 and greater at 1 ng/ml of label concentration. The emission can also be manipulated to last up to 6 seconds or longer. Additional signals, without this list attempting to be limiting in the practice of the present invention, include: triphenyl phosphite-ozone complex, periodates, dioxitanes, bromine and ethanolic alkaline hydrogen peroxide, radioactive isotopes and inorganic or organic peroxides.

### DETAILED DESCRIPTION OF THE INVENTION

As defined herein, a signal comprises a molecule, radical, energy, electromagnetic energy, microwave, ultrasound, radiofrequency discharge, light, chemical, oxidant, singlet oxygen or combination of signals which, when combined with a specific luminescent molecule or a specific luminescence mediating molecule, will cause the production of light. A luminescent label or tag as described herein is a substance bound to analyte, binding partner of analyte, or to ligand of binding partner of analyte either directly (e.g., covalently) or indirectly (e.g., by means of specific binding substance (antibody, protein, etc.), or biotin-avidin or biotin-streptavidin bridge) which when combined with signal either produces light or causes light to be produced. A luminescent label is a luminescent molecule (i.e., the substance which emits light).

As defined herein, a luminescent molecule is a substance, which following singlet oxygen, radiofrequency, electromagnetic energy, light, heat, sound, energy, electric and/or chemical excitation, will emit photon(s) upon decay of orbital electrons to ground state.

As used herein, a luminescent reactant is a free luminescent molecule (i.e., a luminescent molecule that is not bound to analyte, binding partner of analyte or to ligand of binding partner of analyte). Also as used herein, the singular term “luminescent molecule” can also include the plural “luminescent molecules”. Also as used herein, the singular term “luminescence mediating molecule” can also include the plural.

The term asymmetric or asymmetrical, unless otherwise stated, requires only that different substitution exist on the 10 and 10′ positions. The same or different substituent may be present on the various other substitutable positions on the biacridilidene or biacridine oxide (e.g., on the fused benzene rings), but asymmetry must exist as between the 10 and 10′ positions, unless otherwise specifically stated when the term asymmetrical or asymmetric is used.

The term group, as applied to a class of chemical compounds, e.g., alkyl group, refers to not only the literal class (e.g., methyl, ethyl, hexyl, cyclohexyl, iso-octyl, dodecyl, etc.), but also to the substituted counterparts of the class (e.g., hydroxymethyl, 3-chloropropyl, 1,1,1-trifluorohexyl, and the like). Where the terms moiety, species or no qualification are used (e.g., alkyl, alkyl moiety, or alkyl species), the term includes only the literal class, without substitution thereon. Typical substituents which may be used on alkyl and aryl (e.g., phenyl) groups within the practice of the present invention may include, without intending to limit the scope of the invention, alkyl (straight-chain, branched chain, and cycloalkyl) groups, alkoxy groups, halogen (e.g., F, Cl, Br, and I), hydroxyl, carboxylic acid and carboxylate, acetyl, nitro, amino, sulfonate, phosphate, esters, ethers (including thioethers), and other conventional substitutions as known within the art.

The invention is directed to methods for synthesizing asymmetric, uniformly symmetric and symmetric luminescent 10,10′-substituted-9,9′-biacridilidene and biacridine epoxide derivative molecules which can be bound to analyte, binding partner of analyte, or to ligand of binding partner of analyte either directly or indirectly.

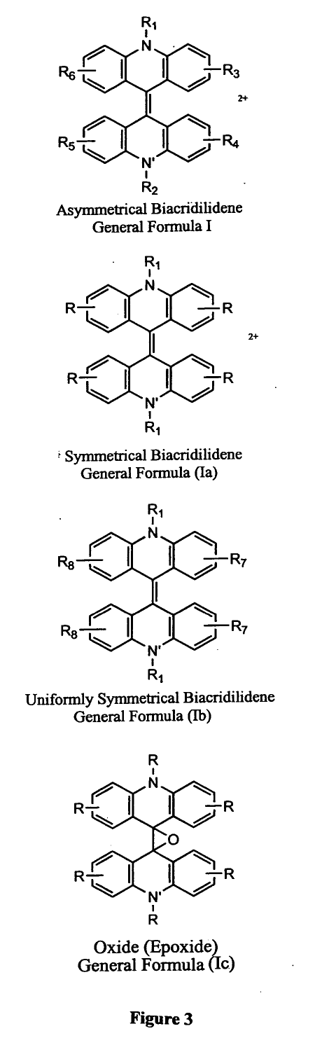

The nature of the symmetrical, uniformly symmetrical or asymmetrical biacridilidene and biacridine epoxide compounds and systems of the present invention may be better appreciated by reference to the formulae for the compounds of the present invention. Formulae I, Ia and Ib for example, show general formulae for 10,10′-substituted-9,9′-biacridilidene compounds having R1 and/or R2 10 and 10′ substituents and R1, R2, R3, R4, R5, R6, R7 and R8 substituents on the non-nitrogen positions (positions 1, 2, 3, 4, 5, 6, 7 and 8, with four positions on each of the fused benzene rings on the central nucleus of the acridines) of the acridine nuclei. Formula Ic is a general formula for 10,10′-substituted-9,9′-biacridene oxide (epoxide) having R substituents on the fused benzene rings of the acridine nuclei (FIG. 3). The compounds are symmetrical according to the present invention when R1 and R2 are identical, irrespective of the nature of groups R3, R4, R5, R6, R7 or R8. It is to be noted that there may be independently more than one of R1, R2, R3, R4, R5, R6, R7 or R8 on the rings. Where R1, R2, R3, R4, R5, R6, R7, or R8 are the same (including hydrogen) and identical with each other, the compound is uniformly symmetrical as defined in the practice of the present invention. For example, the 10,10′-substituted-9,9′-biacridenes of Formulae II, III, IV, V, VI, VII, VIII, IX, X, XVIII, and XIX are uniformly symmetrical, while 10,10′-substituted-9,9′-biacridene compounds XIII, XIV, XV, XVI, and XVII are asymmetrical. These asymmetrical compounds may be most easily formed in mixtures or solutions by reacting two differently substituted acridones (e.g., Formulae XI and XII) together to get a mixture of dimethyl-substituted, di-toluic acid substituted, and monomethyl-monotoluic acid substituted biacridene. They may be separated or purified (as by chromatography). They can also be prepared by other processes and procedures referred to herein.

The invention is also directed to a method for detecting in a sample the presence of a symmetric, uniformly symmetrical or asymmetric 10,10′-substituted-9,9′-biacridilidene and/or biacridine oxide luminescent derivative having specific energy of activation. The method comprises contacting the biacridilidene or biacridene oxide or complexed energy donator(s) (e.g. singlet oxygen generating molecule(s)) with a signal, which comprises at least the energy for overcoming directly or indirectly the specific energy of activation or oxidation potential of the symmetric, uniformly symmetrical or asymmetric 10,10′-substituted-9,9′-biacridilidene and biacridene epoxide. Postulated mechanism for the production of light by symmetric, uniformly symmetrical or asymmetric 10,10′-substituted-9,9′-biacridilidenes or biacridine oxides would begin with the addition of singlet oxygen to the dimer leading to the formation of the dioxetane (moloxide) between 9,9′-carbon atoms. This dioxetane molecule either spontaneously undergoes luminescent decomposition to produce excited state N-methylacridone molecules which produce light as discussed by Janzen, et al., Maeda, et al., and Maskiewicz, et al., supra., or there occurs a reversible luminescent event between biacridilidene and biacridene oxide (Grigoroskii, A. M. and A. A. Simeonov, J. Gen. Chem. (U.S.S.R.), 21:589 (1951)). The excited intermediate emits a photon upon decay of the luminescent molecule to the ground energy state. The light is then measured preferably with a photometric instrument or device such as the Berthold Lumat LB 950 luminometer. This method is more sensitive and more accurate due to lack of interference and self absorption encountered with the methods used to detect fluorophores and phosphors.

Singlet oxygen is preferred for overcoming the energy of activation of the 10,10′-substituted-9,9′-biacridilidene or biacridine oxide. However, singlet oxygen generators, light, heat, energy, radiofrequency discharge, electromagnetic energy, electricity, microwave, ultrasound, chemical, singlet oxygen emitting molecules or a combination of signals are also capable of overcoming the energy of activation of the 10,10′-substituted-9,9′-biacridilidene and biacridine epoxide.

The invention is also directed to luminescent systems for emitting measurable light useful in physical assays, chemical assays, ligand binding assays such as immunoassay or in nucleotide assays. This system comprises an asymmetric, uniformly symmetric or symmetric 10,10′-substituted-9,9′-biacridilidene and/or biacridene epoxide exposed to singlet oxygen having energy of activation bound to analyte or to binding partner of analyte or to ligand of binding partner to analyte, and at least one signal which is/are capable of directly or indirectly overcoming the energy of activation of the 10,10′-substituted-9,9′-biacridilidene and biacridine epoxide. Essentially the 10,10′-substituted-9,9′-biacridilidene or biacridine epoxide acts as label (i.e., a tag, probe, marker, or tracer) and produces light in luminescent reactions. The assay may be direct, homogeneous or heterogeneous and a competitive or sandwich assay or other method of determining the presence of and quantifying the amount of a substance. In a preferred method, the light is produced by the 10,10′-substituted-9,9′-biacridilidene and biacridine epoxide by means of chemiluminescence upon exposure of the 10,10′-substituted-9,9′-biacridilidene, biacridine epoxide and/or associated singlet oxygen generating molecule(s) to signal or signals.

This system is particularly useful for detecting analyte(s) such as a nucleic acid, antibody, antigen, hapten or hapten conjugate, macromolecule, singlet oxygen, cell, particle, bead, protein or polymer. A binding partner to analyte in this system may be nucleotide probe, antibody, singlet oxygen generator, antigen, hapten, hapten conjugate, macromolecule, protein or polymer.

A ligand used herein means a substance, molecule, singlet oxygen generating molecule, linking or binding molecule and may include antigen, antibody, hapten, hapten conjugate, macromolecule, chemical, protein or polymer other than protein such as a polyhydrocarbon, a polyglyceride or a polysaccharide.

A hapten conjugate as used herein is a small molecule (i.e., a molecule having a molecular weight of less than 6,000 Daltons) that is attached to another molecule. An example of a particularly suitable hapten conjugate is a steroid molecule-10,10′-substituted-9,9′-biacridilidene conjugate and/or biacridine epoxide. The analyte may be bound to the binding partner or the binding partner may be bound to the ligand by homobifunctional linker, heterobifunctional linker, biotin-avidin and/or a biotin-streptavidin bridge. The ligand may also be singlet oxygen generating molecule, biotin, avidin or streptavidin and the analyte may also be bound to the biacridilidene and/or biacridine epoxide by means of the biotin-avidin, biotin-streptavidin system. The system provides great sensitivity (up to 10⁻¹⁶ to 10⁻²¹ molar detection of antibody, ligand or antigen) when the system comprises 10,10′-substituted-9,9′-biacridilidene and or biacridine epoxide luminescent label and singlet oxygen as the signal. An even greater sensitivity (up to 10⁻²² molar detection of antibody, ligand or antigen) is obtained when the system comprises a bound singlet oxygen-generating substance (e.g. hypocrelins, hyperricins, phthalocyanines, eosins, porphyrins, etc.).

The chemiluminescent properties of the biacridilidene and/or biacridine epoxide tag together with the other reagents in the system make the system particularly suitable for the development of ultrasensitive assays for many hapten and macromolecular analytes to which the biacridilidene and/or biacridene epoxide associated with singlet oxygen generator can be directly or indirectly conjugated such as hormones, vitamins, toxins, proteins, infectious and contagious agents, chemicals, drugs, tumor markers, receptors, biotin, avidin, streptavidin and genetic material. The biacridilidene and/or biacridine epoxide can also be directly or indirectly conjugated to a specific binding protein such as an antibody for use in chemiluminometric assay development.

The invention is further directed to a chemiluminescent system for emitting measurable light useful in a chemical assay, an immunoassay, a ligand binding assay or a nucleic acid assay which comprises a biacridilidene and/or biacridine epoxide having a specific energy of activation, bound to an analyte, or to a binding partner of an analyte or to a ligand to a binding partner to an analyte and a signal which comprises singlet oxygen, singlet oxygen generator, light, heat, electricity, electromagnetic energy, radiofrequency discharge, microwave, energy, ultrasound, photoactive substance, porphyrin, and fluorophore.

Examples of luminescent molecules for use with the signal in this invention are the cyclic diacyl hydrazides (e.g. luminol and isoluminols), hypocrelins, hyperricins, eosins, porphyrins, cyanines, cera mides and other photoactive compounds.

Again, this chemiluminescent system lends itself to chemical, heterogeneous and homogeneous assays including competitive and sandwich immunoassays. The sensitivity of the system is extremely high when signal comprises singlet oxygen and/or singlet oxygen generating molecule(s) as described above. Again, the analyte may be nucleic acid, antigen, antibody, hapten, hapten conjugate, macromolecule, protein or polymer. The homogeneous assay would involve the use of inhibitors of label luminescence such as polyions. A polycation such as poly(4-vinylpyridinium dichromate) would inhibit, for example, an unbound deuteroporphyrin IX dihydrochloride (DPIX) labeled compound while a polyanion such as poly(vinylalkyl) would inhibit an unbound positively charged biacridilidene and/or biacridine epoxide labeled compound. Unbound in this instance of an assay means that if, for example, the compound is an antigen-label conjugate, it is not bound to, for example, an antibody or if the compound is an antibody-label conjugate it is not bound to an antigen, etc.

The invention is also directed to a method for using symmetrical, uniformly symmetrical or asymmetrical 10,10′-substituted-9,9′-biacridilidene and/or biacridine epoxide in a chemiluminescent heterogeneous assay for detecting the presence of multiple analytes in a sample. Suitable analytes for detection are nucleic acids, antibodies, antigens, haptens, hapten conjugates, macromolecules, polymers or proteins. Again, the method can be a chemical assay, a nucleotide, assay or a ligand binding assay such as an immunoassay. The method may also be a combination of any of these assays. The invention involves the conjugation of biacridilidene and/or biacridine epoxide tag to first analyte to a binding partner of that analyte or to ligand of a binding partner of that analyte and the binding of different tag or label such as nonmetallic tetrapyrrole luminescent molecules or molecules that mediate chemiluminescence such as enzyme, to other analyte or to binding partners of other analyte or to ligands of binding partners of the other analytes. The analytes may be polynucleotide strands, cell, cell constituent, particle, chemical, chemically photoactive compounds such as tetrapyrroles, immunologically active compounds such as antibody, antigen, hapten, hapten conjugate, macromolecule, protein or polymer.

Generally, in the multiple analyte sandwich-type immunoassay, a binding partner to one site on the first analyte is attached to a solid phase such as glass, microbead, particle, cell, polypropylene, polycarbonate or polystyrene and the, thus, coated solid phase is contacted with the sample and second binding partner for a second site on the analyte. The second binding partner is conjugated to the label (e.g., the biacridilidene and/or biacridine epoxide derivative). The same situation exists for the other analyte(s) only the label(s) and the binding partner(s) are naturally different. The solid phase is washed and the bound conjugates are exposed to the appropriate signal, or delivered directly into contact with singlet oxygen and/or singlet oxygen generator(s) (e.g. as in the case of flow cytometry).

Generally, in a competitive assay, the solid phase is coated with limited concentration of binding partner(s) specific for analyte(s) of interest. The solid phase is then contacted with the sample(s) and with a measured amount of first analyte conjugated to the biacridilidene and/or biacridine epoxide and/or associated singlet oxygen generating molecule(s) and with a measured amount of other analyte(s) conjugated to the other luminescent label(s). Following contact, the solid phase is washed to remove any unbound conjugate. With both the sandwich-type or competitive-type assay, the washed solid phase may be separately treated first with signal specific for only one of the labels wherein the label and the signal react to produce emitted light and the presence and/or amount of analyte related to that specific label may be determined by measuring the amount of light emitted, the solid phase can then be separately contacted with other chemiluminescent signal specific for the other label(s) or tag(s) relating to the other analyte(s) whereby those label(s) and signal(s) react to produce emitted light. Again, the measurement of the light from the other reaction(s) will determine the presence and/or amount of other analyte(s) present in the sample.

Since the light produced as a result of different labels has different properties (i.e., the wavelength of light given off by means of each label may differ or the actual amount of light produced per second of reaction may differ between the label(s)), it is possible to treat the solid or liquid phase with signal which will produce light by multiple conjugates simultaneously, differentiate that light and measure the light to determine the amount of each analyte in the sample. One can differentiate the light given off as a result of the different labels by utilizing time resolved luminescent analysis such as that used in fluorometry (Lovgren, T. and K. Pettersson, Luminescence Immunoassay and Molecular Applications, Van Dyke, K. and R. Van Dyke eds., CRC Ress, Boca Raton, Ann Arbor, Boston, Mass., pp. 233-254 (1990)).

The differences in emission properties such as wavelengths can also be utilized (Kleinerman, M. et al., Luminescence of Organic and Inorganic Materials, Kallmann, H. P. and G. M. Spruch eds., International Conference, New York University Washington Square, sponsored by Air Force Aeronautical Research Laboratory, Army Research Office, Curham Office of Naval Research, N.Y.U., pp. 197-225 (1961)).

Preferred luminescent labels for multiple analyte assays with biacridilidene and/or biacridine epoxide derivatives are fluorophores, acridinium derivatives such as acridinium amides, acridinium esters, metallic tetrapyrroles and nonmetallic tetrapyrroles but several other luminescent labels previously discussed are also suitable. A preferred nonmetallic tetrapyrrole is DPIX. The preferred signal solution for producing emitted light by means of tetrapyrroles comprises at a pH from about 10.0 to about 14.0, trans, trans-5-(4-Nitrophenyl)-2,4-pentadienal, sodium di-2-ethylhexyl sulfosuccinate, the luminescent reactant luminol, glucose, benzyltrimethylammonium hydroxide, cumene hydroperoxide, trisodium para periodate, potassium superoxide and EDTA. The signal best suited for flashing the bound 10,10′-substituted-9,9′-biacridilidene and acridinium derivatives comprises at a pH from about 10.0 to about 14.0 in 0.02 borax, EDTA, DMSO, D(-) fructose, potassium superoxide, 2-methyl-2-propanol and aqueous sodium tetraborate. If one of the analyte conjugates is an enzyme labeled analyte conjugate, substrate for that enzyme can be included in the system.

In addition, the invention is directed to a chemiluminescent homogeneous assay for detecting multiple analytes in a sample. In a competitive-type assay, the solid phase is coated with a binding partner specific for each different analyte. The solid phase may additionally be coated with a luminescent reactant. The thus coated solid phase is then contacted with the sample, with a known amount of one of the analytes conjugated to a biacridilidene and/or biacridene epoxide label or associated singlet oxygen generating molecule(s), with known amount of other analyte(s) conjugated to luminescent label(s) other than the biacridilidene and/or biacridene epoxide with polyion(s) (such as poly-N-ethyl-4-vinylpyridinium bromide, poly-4-vinylpyrimidinium dichromate, polyvinylchloride, poly(vinylalcohol), or poly(vinylbenzyl chloride) capable of inhibiting unbound luminescent label conjugate(s) such as anti-TSH-10,10′-biacridilidene and/or biacridene epoxide by preventing the overcoming of the energy of activation of the luminescent label of the unbound conjugate(s) (Vlasenko, S. B. et al, J. Biolum. Chemilum 4:164-176 (1989)). In an assay where it is necessary to inhibit unbound luminescent label antibody conjugate such as DPIX-antibody conjugate a polycation can be used. Following contact, the solid phase is then treated with a signal capable of either producing emitted light by means of multiple conjugates simultaneously or separately contacting the solid phase with a signal specific for one or more label and measuring the emitted light and then separately contacting the solid phase with signal specific for emitting light by means of the label(s) of the other conjugate(s).

The invention is additionally directed to a chemiluminescent signal. The preferred signal is singlet oxygen. The preferred luminescent molecules for use as labels in conjunction with this signal solution are the acridinium and 10,10′-substituted-9,9′-biacridilidene derivatives. However, isoluminol alone and deuteroporphyrin IX.2HCl in conjunction with the luminescent reactant luminol when used with the KO₂ signal solution are also suitable labels. When the singlet oxygen signal is reacted with a luminescent molecule such as biacridilidene and/or biacridene epoxide or a derivative thereof or a luminescent label conjugate such as estradiol 17β-biacridilidene or estradiol 17β-biacridene epoxide, a signal to noise photon emission ratio of at least 20:1 at 1 ng/ml of label concentration is produced for at least 10⁻¹⁵ seconds. Depending upon the label the signal ratio can be 50:1, 100:1 or 200:1 and greater at 1 ng/ml of label concentration using this signal solution and a variety of biacridine labeled conjugates. The emission can also be manipulated to last from up to 6 seconds or longer.

The invention further describes a luminescent signal. The preferred signal is singlet oxygen, which can trigger biacridilidene and/or biacridine epoxide derivative luminescence producing a significant increase in light output, and a change in light output kinetics from the output obtained with previously known signals. When this signal is reacted with luminescent label or luminescent label conjugate such as biacridilidene-antibody, biacridilidene-fluorophore, anti-TSH-biacridene epoxide or biacridene epoxide-fluorophore, a signal to noise photon emission ratio of at least 500:1 at 1 ng/ml of label concentration is produced for at least 10⁻¹⁵ seconds. Again, the signal ratio can be 50:1, 100:1 and even 700:1 and greater at 1 ng/ml of label concentration depending upon variations in the signal and the particular labeled conjugate. The emission can also be manipulated to last from up to 6 seconds or longer. The emission wavelength can also be manipulated through the tranxfer of energy from excited biacridilidene and/or biacridene epoxide to associated fluorophore by means of Forester resonance energy transfer (FRET) (Cochran, L. W. and Forester, D. W., Phys. Rev., 126: 1785 (1962)).

In addition, the invention is directed to a signal, which comprises singlet oxygen. It is preferred in all aspects of the invention where this signal is used that it be contacted with biacridilidene, biacridine epoxide, biacridilidene conjugate, biacridine epoxide conjugate, binding partner of biacridilidene and/or biacridine epoxide or ligand of binding partner of biacridilidene and/or biacridine epoxide. When this signal is reacted with a luminescent label such as a substituted biacridilidene and/or biacridine epoxide, the luminescent reactant produces a signal to noise photon emission ratio of at least 500:1 at 1 ng/ml of luminescent label. Again, depending upon the label the signal to noise ratio can be 50:1, 100:1, 200:1, etc. at 1 ng/ml of label concentration. The emission can also be manipulated to last from up to 6 seconds or longer.

The signal can be any added compound that is capable of overcoming the inherent energy of activation of substituted biacridilidene and/or biacridine epoxide to create a flash or other emission of light. Singlet oxygen is preferred but other oxidants or combination of signals such as hydrogen peroxide, electric discharge, electromagnetic energy, radiofrequency discharge, energy, microwave, ultrasound, heat, sodium perborate, triphenyl phosphite-ozone and bromine and ethanolic hydrogen peroxide are alternatives. These signals can also work alone to overcome the oxidation potential. Singlet oxygen is the preferred signal to produce light from biacridilidenes and/or biacridine epoxides.

The invention now being fully described, it will be apparent to one of ordinary skill in the art that many changes and modifications can be made thereto without departing from the spirit or scope of the appended claims.

### EXAMPLE 1

### Synthesis of 10,10′-Bis[(4-carboxyphenyl)methyl]-9,9′biacridinium dinitrate (Formula XVIII). Biacridilidene and Biacridine Oxide Intermediates Thereof, 10,10′-Bis[(4-N-succinimidyloxycarbonyl)methyl]-9,9′biacridinium dinitrate (Formula XIX) and Antibody Coupling Thereto

This approach to the synthesis of derivatized luminescent biacridine molecules included the synthesis of acridone by the cyclization of diphenylamine-2-carboxylic acids and N-benzoyl-diphenylamine-2-carboxylic acids as described by Acheson and Orgel, supra. (All chemicals and solvents can be obtained from Sigma/Aldrich, St. Louis, U.S.A. and Pacific Pac Inc., Hollister, Calif.). Acridone can also be purchased from Sigma/Aldrich.

In addition to the preparation of acridone, a methyl ester of a substituent molecule was synthesized for covalent attachment at the 10-nitrogen atom of acridone. A substituent molecule for derivatizing a biacridine molecule as used herein is a molecule having a functional group that provides for the further derivatization of the biacridine or for the attachment of the biacridine to other molecules such as antigens, antibodies, etc. Usually the substituent molecule used in the course of the invention has a molecular weight of about 10,000 or less. In this example, the methyl esterification of the substituent molecule alpha-bromo-para-toluic acid was carried out. Other molecules having good leaving group(s) (such as halogen atom(s)) at one end of the molecule and the presence of functional group(s) elsewhere on the molecule, can also serve as substituent molecules and be successfully esterified. A good example of another substituent molecule of this type is iodoacetic acid. Esterification was accomplished by reacting the substituent molecule in 10% boron trifluoride in methanol (25 ml of boron trifluoride-methanol per gram of substituent molecule was preferably added). This was allowed to react for at least 10 hours at room T° and the methyl ester was extracted with methylene chloride in a separation funnel. The extract was washed twice with H₂O, once with 0.1 M sodium bicarbonate and twice again with H₂O. The volume was reduced to dryness at 60° C. on a rotavapor RE120 rotary evaporator. Alternate methods of methyl ester synthesis are the use of ether followed by the addition of diazomethane and the use of methanol containing 5% concentrated sulfuric acid.

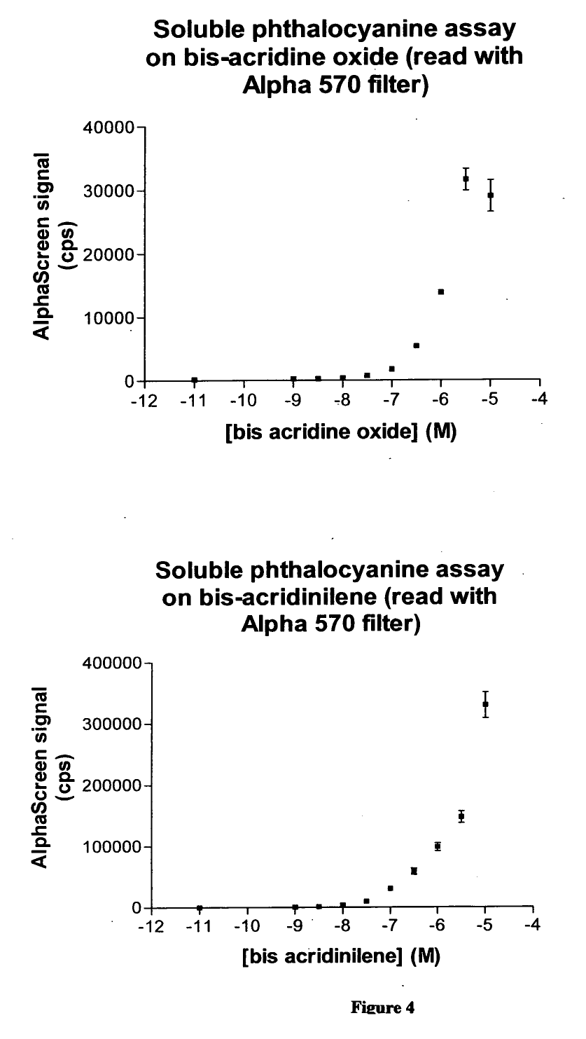

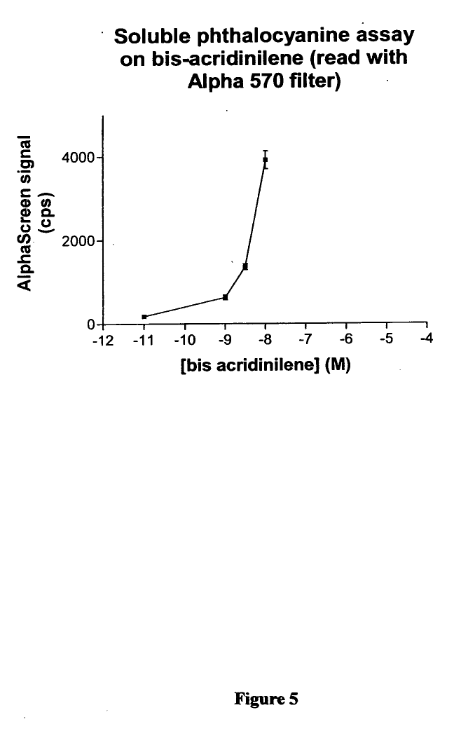

The next step in the synthesis was the alkylation of acridone. To accomplish this, 5.4 mM of acridone and 6.5 mM of sodium hydride were added to 100 ml anhydrous tetrahydrofuran (THF). This mixture was then refluxed with stirring for 2 hours at 70° C. under argon gas. To this mixture was added 5.5 mM of the substituent methyl ester (e.g., alpha-bromo-para-toluic acid methyl ester) and the combination was refluxed with stirring at 70° C. for 10-13 hours. Silica gel thin layer chromatography (Baker Chemical Co., Phillipsburg, Pa.) at this time with 2% methanol in methylene chloride revealed a spot with R_{f}=0.2 for hydrolyzed acridone-10-substituent; a second spot at R_{f}=0.4 for unreacted acridone; a third spot (very small) at R_{f}=0.5 for unknown byproduct; a fourth spot at R_{f}=0.6 for the acridone-10-substituent methyl ester (acridone-10-para-toluic acid methyl ester); and a fifth spot at R_{f}=0.9 for unreacted substituent-methyl ester. The reaction mixture was a light lemon-brown color containing a precipitate (ppt.). The ppt. (containing mostly hydrolyzed acridone-10-substituent) was filtered off and discarded. The filtrate was extracted with ethyl acetate and water in a separation funnel to remove remaining salts and hydrolyzed material. The impurities remained in the aqueous phase. The volume of the organic phase (ethyl acetate phase) was reduced to dryness on a rotaevaporator at 60° C. Methylene chloride was then added and the unreacted (insoluble) acridone precipitate was filtered off. The methylene chloride extract was then eluted and purified on a silica-60 column with 3% ethyl acetate in methylene chloride, with the acridone-10-para-toluic acid methyl ester being eluted and contained within the first yellow band. This material was again concentrated on a rotaevaporator with replacement of the ethyl acetate-methylene chloride eluant by methanol (through a continuous feed tube on the rotaevaporator). The purified acridone-10-substituent methyl ester precipitated as a light yellow crystalline material. This precipitate was filtered and washed with methanol (yield approximately 50%). The product is kept dry and free of oxygen by storage under argon gas atmosphere. These molecules and their acid precursors were active fluorophores with (for examples) acridone-10-para-toluic acid and its NHS derivative exciting at 403 nm and emitting at 440 nm; and acridone-10-acetic acid and its NHS derivative exciting at 398 nm and emitting at 438 nm. The acridone-10-substituent methyl ester was then refluxed in phosphorous oxychloride (POCl₃) over oil bath at 120°-130° C. The acridone-10-substituent methyl ester was then dimerized by reacting for 30 minutes in a mixture of 1.76 g zinc dust and 132 ml dry acetone. The intermediate product 10,10′-ρ-toluic acid-9,9′-biacridilidene will luminesce in the presence of singlet oxygen, light, electromagnetic energy, radiofrequency discharge or any energy capable of overcoming the energy of activation of biacridilidene (FIGS. 4 and 5).

Acridone-10-substituted intermediates (e.g., acridone-10-acetic acid) were also directly synthesized by mixing well together 8.45 g of 2-chlorobenzoic acid, 7.80 g N-phenylglycine, 11.00 g anhydrous potassium carbonate and 0.30 g Cu++ powder in 6 ml H₂O. This mixture was then refluxed overnight over an oil bath at 160° C. Ethanol was added slowly and the product was dissolved in water, filtered and ppt. with HCl. The whole mixture was refiltered to remove unconsumed 2-chlorobenzoic acid and the remaining oil in that filtrate allowed to crystallize. The filtrate was dissolved in NaOH, filtered, acetic acid was added and the mixture was refiltered to remove further unreacted 2-chlorobenzoic acid. The product was precipitated by the addition of HCl (acid product) and dried. Then it was extracted with excess benzene and further purified by dissolving in sodium acetate solution, boiling with activated charcoal and reprecipitating with HCl. The pure product was again filtered and crystallized from dilute methanol to give a white ppt. (mp. 165-167° C.).

Acridone-10-substituted molecules (e.g., acridone-10-acetic acid) were also synthesized, by refluxing a mixture of 500 mg (2.7 mM) acridone, 130 mg 80% NaH in mineral oil and 50 ml anhydrous Tetrahydrofuran (THF) under argon for 2-4 hours. Iodoacetic acid (540 mg, 2.7 mM) was then added and refluxing of the mixture was continued under argon for an additional 10 hours. The ppt. was filtered and the filtrate was purified on a reverse phase column under 20-30% ethanol elution. This purified material was then dried and taken up in THF and hydrolyzed with 4 N NaOH for 10 hours. Water was added and the mixture was filtered. The filter was washed with H₂O, and the mixture was brought to a pH of 8.0 with 1 N HCl. Final purification was performed on reverse phase silica gel column with 10 to 30% methanol in water. The volume was reduced on a rotaevaporator (e.g., RE120) and reprecipitation was carried out with 1 N HCl overnight at pH 2.5. The product was collected by centrifugation and washed with water once. It was dried on a lyophilizer (yield approximately 40%).

Conversion of the acridone-10-para-toluic-acid methyl ester to the quaternized 9-chloro-acridine-10-para-toluic acid methyl ester was accomplished by reacting the acridone-10-substituent methyl ester with POCl₃. One milliliter of POCl₃ was added to each 50 mg of the purified methyl ester and this mixture was refluxed for 1 hour over an oil bath at 120° C.

Dimerization and de-esterification of the 9-chloro-acridine-10-para-toluic acid methyl ester was accomplished by the addition of 1 gram of cold zinc metal and 10 ml of freezing cold concentrated HCl/100 mg of 9-chloro-acridine-10-para-toluic acid methyl ester which was allowed to react under freezing conditions for anywhere from 1 to 10 hours. This reaction is violent and must be carried out under freezing conditions for 1-10 hours. The 10,10′-ρ-toluic acid-9,9′-biacridilidene ppt. was then filtered off and washed with water. Purification of the acid filtrate was accomplished on a silica gel C-18 reverse phase column. The column was pretreated with methanol followed by 0.1 N nitric acid followed by 0.01 M phosphate buffer. Elution of the filtrate was first accomplished with methanol in 0.01 M phosphate buffer to remove the byproducts, unreacted materials and salts. The product, (10,10′-para-toluic acid-9,9′-biacridine salt, a disubstituted biacridine) which sticks to the top of the column, was then eluted with 30 to 90% methanol in 0.1 N nitric acid (the methanol strength should be increased from 30% to 90% to remove all product). Product eluted as a yellow band with approximately 50% methanol in 0.1 N nitric acid. The protonated purified dimer-dinitrate salt (10,10′-para-toluic acid-9,9′-biacridinium dinitrate) was then concentrated on a rotaevaporator at 60° C. to a small volume (5 ml) and was lyophilized to dryness. This biacridinium dinitrate product can be converted to 10,10′-ρ-toluic acid-9,9′-biacridine epoxide (oxide) by dissolving the biacridinium dinitrate in ethanol or methanol and adding caustic soda (40-50% sodium hydroxide). This reaction yields a dark greenish-black solution. Yellow-green epoxide crystals form from this solution by washing with methanol and water. Biacridine epoxide (oxide) can also be synthesized through a one electron reduction of the biacridinium diniatrate in the presence of silver dioxide (AgO₂) to yield the intermediate biacridine carbinol diquaternary base. The carbinol base is then converted to the biacridine epoxide (oxide) by the addition of alcohol. The biacridine carbinol diquaternary base can also yield biacridilidene through a one electron reduction in the presence of sodium hydroxide (FIG. 1). Scanning spectrophotometry on a Beckman DU-600 spectrophotometer revealed characteristic biacridinium dinitrate absorbance with a preliminary shoulder at 461.5 nm, a first peak at 438.5 nm, a second shoulder at approximately 415 nm, a major peak at 370.5 nm and a trailing shoulder at approximately 355 nm (see FIG. 2). NMR plot on a Bruker ARX 400 instrument (Rheinstetten-FO, Germany) gave a peak at 6.8 ppm indicating the presence of the methylene carbon attached to the 10 position nitrogen on the dimer and the presence of multiple aromatic carbon peaks in the 7 to 9 ppm range.

Further derivatization of the dimer with N-hydroxysuccinimide (NHS) was accomplished by adding (with stirring) 211 micromoles (uM) of dicyclohexylcarbodiimide to 141 uM of the dimer in dry dimethylformamide (DMF) (0.5 ml/mg of dimer). To this was added 211 uM of N-Hydroxysuccinimide, which was allowed to react at room T° for 10 hours (Formula XIX). Urea precipitates, which formed during this reaction, were filtered off. This NHS-ester of the label is very stable in an amber vial (at least one year). On reverse phase TLC the major peak did not move on elution with 90% methanol in 0.01 M phosphate buffer (a yellow spot at the origin under long wavelength U.V. light), but does move with an R_{f}=0.2 in 0.1 nitric acid/70% methanol (v/v).

Conjugation of the 10,10′-para-toluo-NHS-9,9′-biacridinium dinitrate derivative to antibody began with the addition of 100 microliters of the DMF solution of the derivative to 1 mg of the antibody in PBS at pH 7.4. In this example polyclonal antibody to the beta-chain of thyroid stimulating hormone was conjugated, however, any antibody, analyte, polymer or binding protein can be utilized. This mixture was allowed to react at room T° for 10 hours and then 54 microliters of a 1 mg/ml solution of d-L-lysine was added to the antibody-derivative mix and was allowed to react for an additional 3 hours. This step is necessary for occupying unreacted NHS sites on the luminescent derivative-antibody conjugate.

This antibody-10,10′-substituted-9,9′-biacridine conjugate was purified on a 20 cm Biogel P-10 column (BioRad; Hercules, Calif.) by eluting with a buffer containing 10 mM dibasic potassium phosphate and 0.1 M NaCl at a pH of 7.4. The antibody conjugate eluted in the first fraction off the column, which can be monitored by TLC and spectrophotometry. The antibody conjugate had two spectrophotometric peaks at 365 nm (small peak for the label) and at 275 nm for the antibody and was successfully flashed with the signal solution of the invention described in Example 2.

A mildly acidic environment (0.01 N HNO₃) stabilizes the labels and also gives the strongest signal-to-noise ratio. A wash solution containing 0.2 microliters Tween®-20/ml PSS brought to 0.01 N with HNO₃ should work well in separation-required assays. Exposing the label to 5 microliters of the final wash solution just before flashing may also enhance the signal.

### EXAMPLE 2

### Preparation of Signal Solution

The signal solution for the production of light from the new chemiluminescent molecules was formulated as follows:

To each 100 ml of 0.02 M sodium tetraborate was added the following with stirring:

a) 0.744 mg (0.02 mM) ethylenediaminetetraacetic acid (EDTA)

b) 100 μl of dimethylsulfoxide (DMSO)

c) 400 mg (0.02 M) D(-) fructose

d) 1996 mg (280 mM) potassium superoxide (KO₂)

e) 17 ml 2-Methyl-2-propanol

### EXAMPLE 3

### Assay Comparing Singlet Oxygen Initiated Luminescence of Biacridilidene Bis-ethylene Diamine and Biacridinium Epoxide Sodium Salt

12.5 microliters of a 200 nM solution of zinc tetrasulfonate phthalocyanine in phosphate buffered saline (PBS) pH 7.4 was added in triplicate to 11 columns of wells of a 96 well microtitre plate. Serial dilutions of biacridilidene and biacridinium epoxide ranging from 10⁻⁴ to 10⁻¹¹ M were prepared from a 1 mM stock solution of each in DMSO. Dilutions were made in PBS pH 7.4 and 12.5 μl of each dilution was added to a triplicate of wells containing phthalocyanine, leaving the first triplicate free of biacridilidene and biacridinium epoxide as a zero control. Each plate was read on an EnVision multilabel reader (PerkinElmer, Meriden, Conn.) using the Alpha 570 filter (FIGS. 4 and 5). This instrument uses laser light energy to excite the phthalocyanine molecules which in-turn release singlet oxygen causing light emission from the biacridilidene and biacridinium epoxide molecules.

### EXAMPLE 4

### Assay Demonstrating the Sensitivity of Signal with Increasing Dilutions of Biacridilidene Bis-ethylene Diamine-Labeled Biotinylated Rabbit IgG

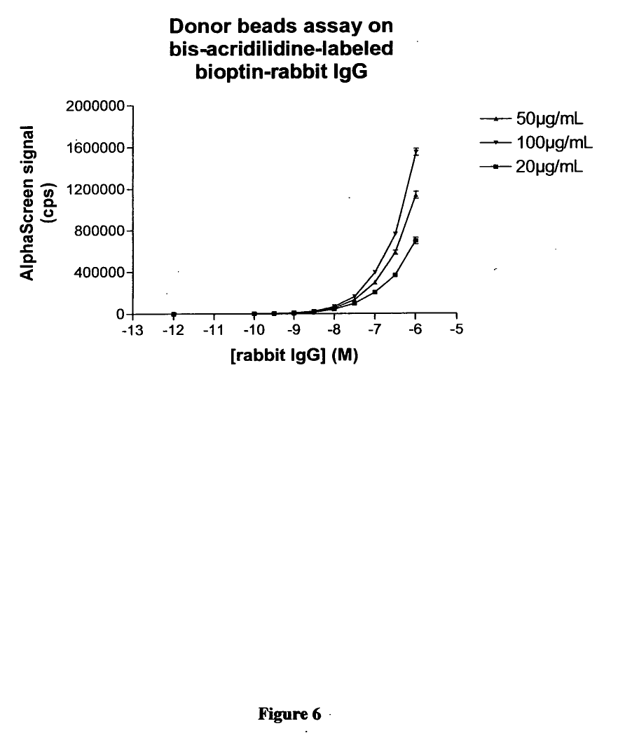

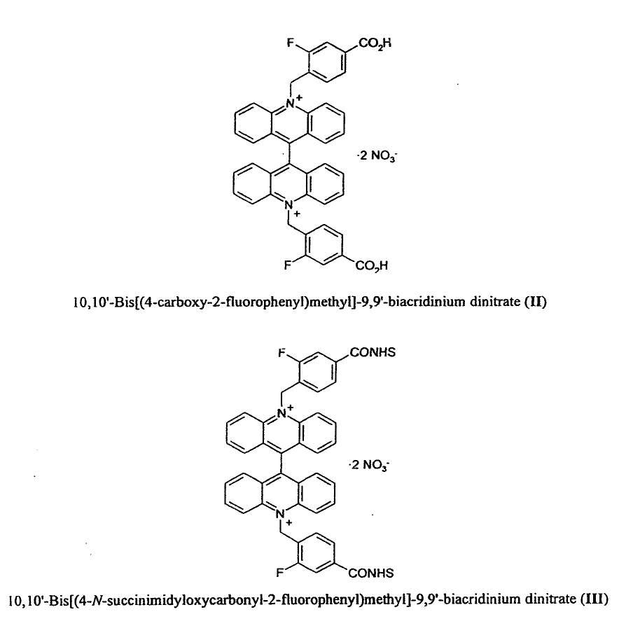

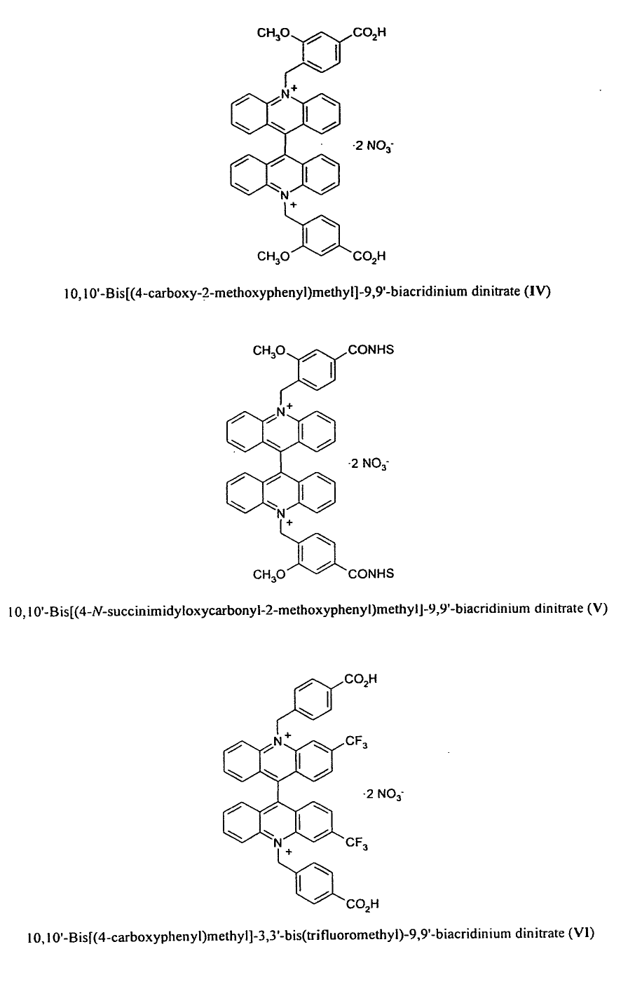

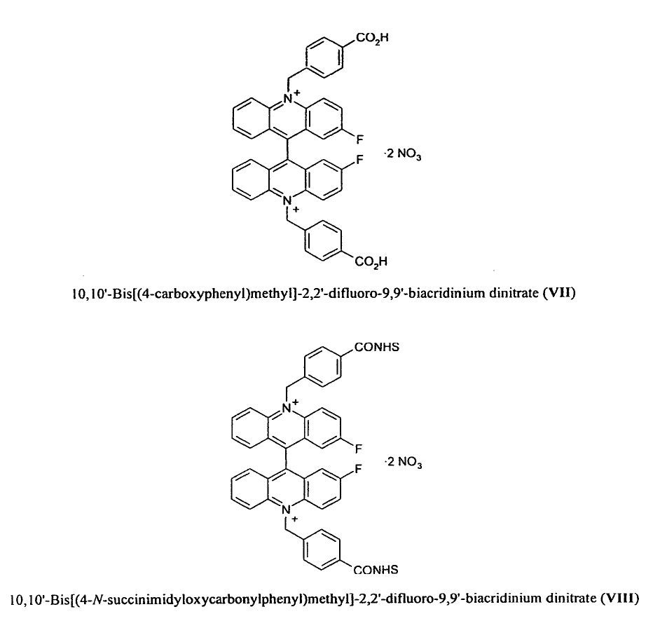

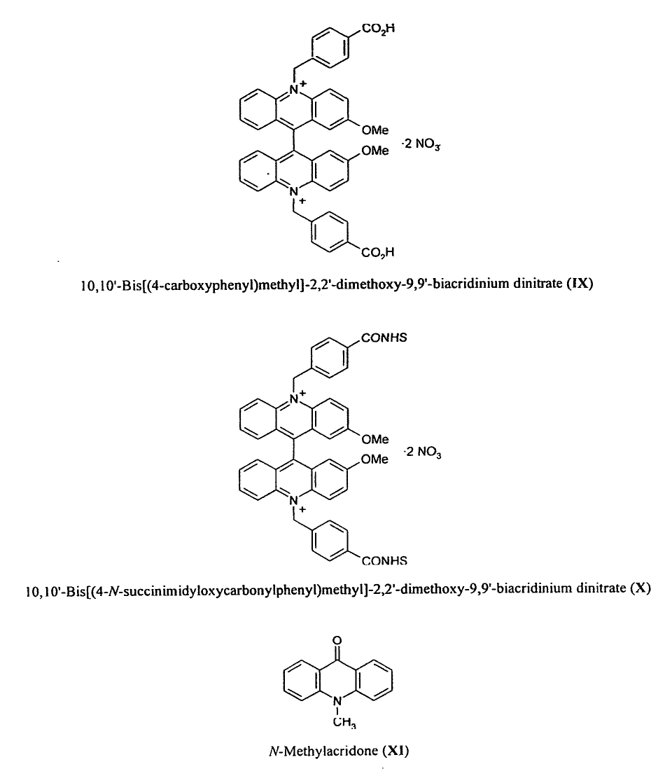

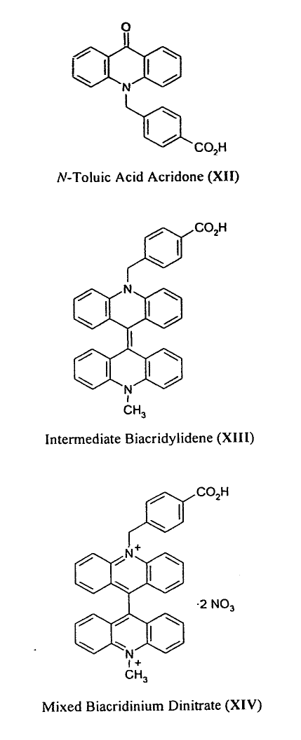

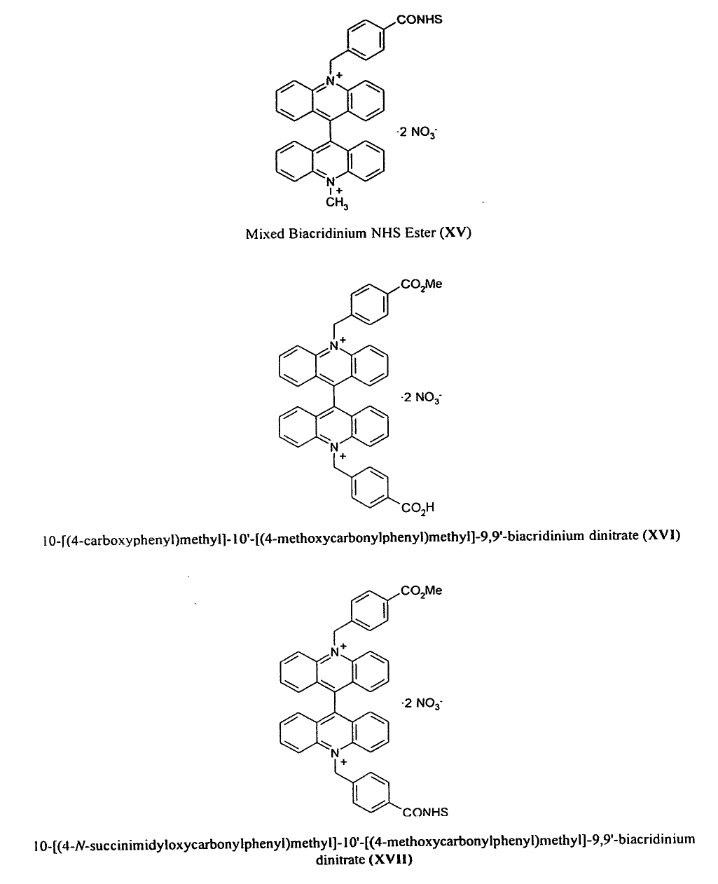

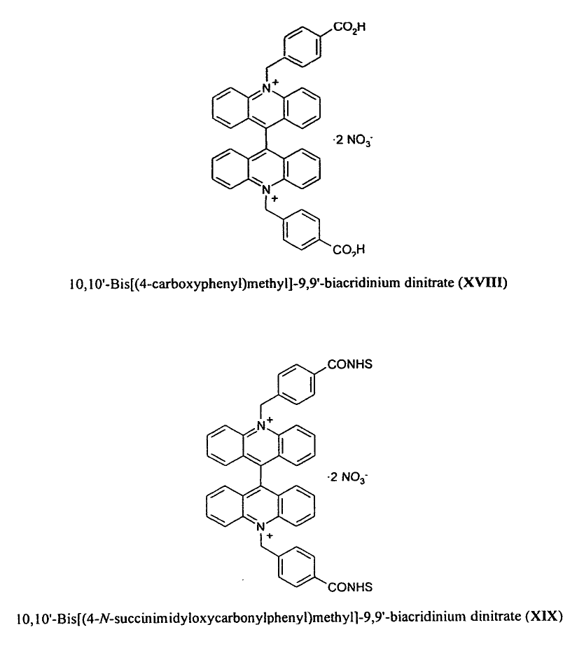

This example demonstrated the luminescent singlet oxygen functionality of biacridilidene-antibody (binding agent) conjugate and the linearity of signal with increasing labeled antibody dilutions. In 500 μl 0.1 M carbonate buffer (pH 8.5) containing 10 mM ascorbic acid, add 200 μg biacridilidene bis-ethylene diamine, 1 mg sulfo-disuccinimidyl tatarate (sulfo-DST, Pierce Chemical Co.), 50 μl DMSO and 10 μl tetrahydrofuran (THF). This mixture was incubated 5 minutes followed by addition of 500 μl of 5 mg/ml biotinylated IgG in PBS (pH 7.4). This mixture was incubated 1 hour at room T°. Purification of the biacridilidene-labeled IgG was performed on a PD10 column. Elution was accomplished using a saturated BSA-purified water solution followed by PBS (pH 7.4). The assay was performed on a 96 well microtitre plate, with a beginning dilution of 1 μM followed by nine 0.5 log dilutions and a zero (no antibody). Dilutions were made in PBS (pH 7.4) and 20 μl/well of each was added in triplicate to the plate wells. To each of these wells was then added 5 μl of phthalocyanine impregnated carboxylated donor beads having streptavidin conjugated to surface using standard carbodiimide coupling chemistry. Three dilutions of streptavidin-phthalocyanined donor beads (20, 50 and 100 μg/ml) were used in triplicate to generate the three curves in FIG. 6. The plate was read on an EnVision multilabel reader (PerkinElmer, Meriden, Conn.) using the Alpha 570 filter.

### EXAMPLE 5

### Bicaridinium Compounds Including: Precursors, Symmetric Substitution on the Toluic Acid Ring and Acridine Ring and Asymmetric Substitution on the Acridine Ring

Materials used: N-methyl acridone (Aldrich, 19,250-3), Acridone (Acros, 40025-0250), Methyl 4-(bromomethyl)-benzoate (Aldrich, 34,815-5), Zinc dust (Fisher Scientific).

Symmetric Substitution on the Toluic Acid Ring

a. Fluoro Substituted
1). 10,10′-Bis[(4-carboxy-2-fluorophenyl)methyl]-9,9′ biacridinium dinitrate (Formula II) and the corresponding NHS ester (Formula III)

b. Methoxy Substituted
2). 10,10′-Bis[4-carboxy-2-methoxyphenyl)methyl]-9,9′ biacridinium dinitrate (Formula IV) and corresponding NHS ester (Formula V)
   Several structural variations of 10,10′-bis[(4-carboxyphenyl)methyl]-9,9′-biacridinium dinitrate were prepared using similar synthetic techniques. These compounds contained substituents on the toluic acid appendage or on the biacridinium core. N-Hydroxysuccinimide (NHS) esters were prepared for the two derivatives with substituents on the toluic acid appendage.

The substituted-toluic acid biacridinium compounds are prepared by the alkylation of 9(10H)-acridone with a methyl 4-(bromomethyl)-3-substituted-benzoate. The methyl 4-(bromomethyl)-3-substituted-benzoates are commercially available (3-methoxy), or readily available using literature procedures (3-fluoro). The methyl esters of the alkylated acridones are saponified with methanolic sodium hydroxide to afford the respective sodium salts of the acids. These salts are dimerized using zinc and concentrated hydrochloric acid, and the intermediate dimers are oxidized with dilute nitric acid to give the substituted-toluic acid biacridinium compounds. The NHS esters of these acids are obtained by coupling the acid with NHS in the presence of N,N′-dicyclohexylcarbodiimide.

### Preparation of 10,10′-Bis[(4-carboxy-2-fluorophenyl)methyl]-9,9′-biacridinium dinitrate (Formula II) and 10,10′-Bis[(4-N-succinimidyloxycarbonyl-2-fluorophenyl)methyl]-9,9′-biacridinium dinitrate (Formula III):

Methyl 3-fluoro-4-methylbenzoate. A mixture of 3-fluoro-4-methylbenzoic acid (9.94 g, 61.3 mmol) and methanol (60 mL) was treated with a solution of 95% sulfuric acid (5.72 g, 55.4 mmol) in methanol (40 mL). The reaction mixture was heated at reflux for 1.5 hours. The mixture was cooled, poured into 100 mL of water, and extracted with ethyl ether (3×50 mL). A small amount of solid sodium chloride was added to facilitate phase separation. Evaporation of the solvent on a rotary evaporator gave a very pale yellow liquid (9.66 g, 94%).

Methyl 4-(bromomethyl)-3-fluorobenzoate. To a stirred mixture of N-bromosuccinimide (6.75 g, 37.5 mmol) in carbon tetrachloride (100 mL) was added a solution of methyl 3-fluoro-4-methylbenzoate (6.31 g, 37.5 mmol) in carbon tetrachloride (50 mL) and 2,2′-azobisisobutyronitrile [AIBN] (32 mg, 0.2 mmol). The reaction mixture was heated at 75° C. for 3 hours. To the flask was added additional AIBN (31 mg, 0.2 mmol) and the mixture was heated at reflux (˜80° C.) for 2.5 hours. The solids were filtered and rinsed with carbon tetrachloride. The filtrate was washed with 10% sodium thiosulfate (2×15 mL), saturated sodium bicarbonate (15 mL), and dried over sodium sulfate. Evaporation of the solvent on a rotary evaporator, followed by evacuation under high vacuum, gave a pale yellow oil (8.67 g). This liquid was purified by flash chromatography on a silica gel column eluted with 95:5 hexanes:ethyl acetate to afford a very pale yellow liquid (4.70 g, 51%). (Where the term ‘hexanes’ is used, the term means that the solvent is a mixture of hexane isomers).

10-[(4-Carboxy-2-fluorophenyl)methyl]-9(10H)-acridone sodium salt. To a stirred suspension of sodium hydride (0.84 g, 21.0 mmol) in tetrahydrofuran (45 mL) and dimethyl sulfoxide (12 mL) was added 9(10H)-acridone (3.33 g, 16.9 mmol) in portions over 10 min. The mixture was stirred at ambient temperature for 0.5 hours. To the resulting green solution was added a solution of methyl 4-(bromomethyl)-3-fluorobenzoate (4.69 g, 18.6 mmol) in tetrahydrofuran (5 mL). The mixture was heated for 2 hours at 69° C. and cooled to ambient temperature. The mixture was treated with acetic acid (0.56 g, 9.3 mmol) and stirred for 15 min. The solvent was concentrated on a rotary evaporator. The concentrate was diluted with water, ethyl acetate, and hexanes (65 mL each). The mixture was swirled and the resulting solid was collected by suction filtration. The filter cake was rinsed with water and ethyl acetate (25 mL). The solid was dried to give a light yellow powder (5.25 g, 86%).

A portion of this powder (2.50 g, 6.9 mmol) was suspended in methanol (13 mL). The suspension was treated with a solution of sodium hydroxide (0.54 g, 13.1 mmol) in water (22 mL). The mixture was heated at reflux for 2.5 hours, cooled to ambient temperature, and then in an ice-water bath for 0.5 hours. The resulting solid was collected by suction filtration. The filter cake was rinsed with water, acetone, ethyl acetate, and hexanes (5 mL each). The yellow powder was stirred with acetone (19 mL) for 2.3 hours and the solid was collected by suction filtration. The filter cake was rinsed with acetone and dried to give a pale yellow powder (2.37 g, 93%).

10,10′-Bis[(4-carboxy-2-fluorophenyl)methyl]-9,9′-biacridinium dinitrate (II). To a stirred suspension of 10-[(4-carboxy-2-fluorophenyl)methyl]-9(10H)-acridone sodium salt (2.29 g, 6.20 mmol) in acetone (52 mL) was added zinc dust (6.79 g, 103 mmol). The mixture was stirred for 15 min at ambient temperature. The mixture was cooled to 12° C. in a cold water bath and 37% hydrochloric acid (61.15 g, 621 mmol) was added dropwise over 4 hours, maintaining the reaction temperature at 20±2° C. during the addition. After stirring for 1 hour (h) at ambient temperature, some zinc remained in the reaction mixture. Additional 37% HCl was added in portions (1.28 g, 13 mmol, 1.47 g, 15 mmol; 2.94 g, 30 mmol) at 0.5 hour intervals. The zinc was consumed after an additional 1.5 hours and the mixture was stirred overnight. The bright yellow solid was collected by suction filtration and rinsed thoroughly with water. The filter cake was rinsed with acetone, ethyl acetate, and hexanes (5 mL each), and dried briefly. The solid was suspended in acetone and treated with 6% nitric acid (55.50 g, 52.8 mmol). The mixture was heated at 70° C. for 2 hours, the color turning from yellow to light orange. Upon cooling, the solid was collected by suction filtration and rinsed with water. The filter cake was rinsed with acetone, ethyl acetate, and hexanes (5 mL each) and dried. The solid was further dried on a rotary evaporator at 50° C. for 1.5 hours to afford a light orange powder (2.30 g, 94%).

10,10′-Bis[(4-N-succinimidyloxycarbonyl-2-fluorophenyl)methyl]-9,9′-biacridinium dinitrate (III). A solution of 10,10′-bis[(4-carboxy-2-fluorophenyl)methyl]-9,9′-biacridinium dinitrate (II) (520 mg, 0.66 mmol) in N,N-dimethylformamide (DMF, 12 mL) was treated with N-hydroxysuccinimide (NHS, 206 mg, 1.79 mmol) and N,N′-dicyclohexylcarbodiimide (DCC, 310 mg, 1.50 mmol). The solids were rinsed in with DMF (4 mL). The mixture was stirred at ambient temperature for 20.5 hours. To the reaction was added NHS (24 mg, 0.21 mmol) and DCC (58 mg, 0.49 mmol) and stirring was continued for 5.75 hours. The reaction mixture was diluted with acetonitrile (8 mL) and stirred for 25 min. The solids were removed by suction filtration and rinsed thoroughly with acetonitrile. The filtrate was concentrated on a rotary evaporator and the concentrate was poured slowly into rapidly stirred ethyl acetate (100 mL). The resulting yellow solid was stirred for 15 min. and collected by suction filtration. The filter cake was rinsed with ethyl acetate and hexanes and dried to give a yellow powder (0.43 g, 66%).

### b. Preparation of 10,10′-Bis[(4-carboxy-2-methoxyphenyl)methyl]-9,9′-biacridinium dinitrate (Formula IV) and 10,10′-Bis[(4-N-succinimidyloxycarbonyl-2-methoxyphenyl)methyl]-9,9′-biacridinium dinitrate (Formula V):

10-[(4-Carboxy-2-methoxyphenyl)methyl]-9(10H)-acridone sodium salt. To a stirred suspension of sodium hydride (1.50 g, 37.5 mmol) in tetrahydrofuran (80 mL) and dimethyl sulfoxide (20 mL) was added 9(10H)-acridone (5.92 g, 30.0 mmol) in portions over 20 min. The mixture was stirred at ambient temperature for 0.5 hours. To the resulting green solution was added methyl 4-(bromomethyl)-3-methoxybenzoate (8.33 g, 33.0 mmol) as a solid in one portion. The mixture was heated for 4.75 hours at 65-70° C. and cooled to ambient temperature. To the flask was added sodium hydride (0.15 g, 3.75 mmol) and methyl 4-(bromomethyl)-3-methoxybenzoate (0.40 g, 1.05 mmol). The reaction was heated at 65-70° C. for 4 hours and cooled to ambient temperature. The mixture was treated with acetic acid (0.93 g, 15.5 mmol) and stirred for 0.5 hours. The reaction mixture was partitioned between water, ethyl acetate, and hexanes (115 mL each). A solid crystallized upon standing. The resulting solid was collected by suction filtration and rinsed with water and ethyl acetate. Additional solid separated in the filtrate and the filtrate was re-filtered twice. The organic layer of the filtrate was evaporated on a rotary evaporator to give a moist, olive-green solid (9.58 g). The filter cake was dried to give a light yellow powder (4.97 g). These two solids were combined with ethyl acetate (12 mL) and hexanes (108 mL). The solid was collected by suction filtration. The filter cake was rinsed with hexanes and dried to afford a light yellow-green powder (11.23 g, 100%).

A portion of this powder (5.46 g, 14.6 mmol) was suspended in methanol (27 mL) and water (10 mL). The mixture was heated for 2.6 hours at 75-80° C., cooled to ambient temperature, and then cooled in an ice-water bath for 2 hours. The resulting solid was collected by suction filtration. The filter cake was rinsed with ice-cold water, acetone, ethyl acetate, and hexanes (10 mL each). The yellow powder was stirred with acetone (37 mL) for 1 hour and the solid was collected by suction filtration. The filter cake was rinsed with acetone and dried to give a light yellow powder (4.70 g, 84%).

10,10′-Bis[(4-carboxy-2-methoxyphenyl)methyl]-9,9′-biacridinium dinitrate (IV). To a stirred suspension of 10-[(4-carboxy-2-methoxyphenyl)methyl]-9(10H)-acridone sodium salt (2.29 g, 6.00 mmol) in acetone (50 mL) was added zinc dust (13.05 g, 198 mmol). The mixture was stirred for 15 min at ambient temperature. The mixture was cooled to 12-13° C. in a cold water bath and 37% hydrochloric acid (119.42 g, 1212 mmol) was added dropwise over 6 hours, maintaining the reaction temperature at 20±2° C. during the addition. The mixture was stirred overnight at ambient temperature. The bright yellow solid was collected by suction filtration and rinsed thoroughly with water. The filter cake was rinsed with acetone, ethyl acetate, and hexanes (5 mL each), and dried briefly. The solid was treated with 6% nitric acid (155.68 g, 148.2 mmol). The mixture was heated for 2.3 hours at 70-80° C., the color turning from yellow to light orange. Upon cooling, the solid was collected by suction filtration and rinsed with water. The filter cake was rinsed with acetone, ethyl acetate, and hexanes (5 mL each) and dried. The solid was further dried on a rotary evaporator at 50° C. for 1.5 hours to afford a light orange powder (1.78 g, 73%).

10,10′-Bis[(4-N-succinimidyloxycarbonyl)-2-methoxyphenylmethyl]-9,9′-biacridinium dinitrate (V). A solution of 10, 10′-bis[(4-carboxy-2-methoxyphenyl)methyl]-9,9′-biacridinium dinitrate (IV) (536 mg, 0.66 mmol) in N,N-dimethylformamide (12 mL) was treated with N-hydroxysuccinimide (229 mg, 1.79 mmol) and N,N′-dicyclohexylcarbodiimide (328 mg, 1.59 mmol). The solids were rinsed in with DMF (4 mL). The mixture was stirred at ambient temperature for 21 hours. The reaction mixture was diluted with acetonitrile (8 mL) and stirred for 0.5 hours. The solids were removed by suction filtration and rinsed thoroughly with acetonitrile. The filtrate was concentrated on a rotary evaporator and the concentrate was poured slowly into rapidly stirred ethyl acetate (80 mL). The evaporation flask was rinsed with ethyl acetate (20 mL). The resulting yellow solid was stirred for 0.5 hours and hexanes (10 mL) was added. The solid was collected by suction filtration. The filter cake was rinsed with ethyl acetate and hexanes and dried to give a yellow powder (0.41 g, 62%).

Symmetric Substitution on the Acridine Ring

a. Fluoro Substituted
1). 10,10′-Bis[(4-carboxyphenyl)methyl]-3,3′-bis-(trifluoromethyl)-9,9′-biacridinium dinitrate (Formula VI)
2). 10,10′-Bis[4-carboxyphenyl)methyl]-2,2′-difluoro-9,9′-biacridinium dinitrate (Formula VII) and the corresponding NHS ester (Formula VIII).

b. Methoxy Substituted
1). 10,10′-Bis[(4-carboxyphenyl)methyl]-2,2′-dimethoxy-9,9′-biacridinium dinitrate (Formula IX) and corresponding NHS ester (Formula X).

Fluorinated Biacridine Compounds

The acridine ring-substituted biacridinium derivative was prepared starting with N-[3-(trifluoromethyl)phenyl]anthranilic acid (Pellón, R. F., et al. Tetrahedron Lett. 1997, 38, 5107-5110). The acid was cyclized to a mixture of 1- and 3-(trifluoromethyl)-9(10H)-acridones using polyphosphoric acid (Metz, G Synthesis, 1972, 612-614). A pure sample of the 3-(trifluoromethyl)-9(10H)-acridone was isolated by flash chromatography. This acridone was alkylated with methyl 4-(bromomethyl)benzoate and the ester was saponified with methanolic sodium hydroxide. The resulting acid was dimerized with zinc and concentrated hydrochloric acid and the intermediate dimer was oxidized with dilute nitric acid to afford the trifluoromethyl-substituted biacridinium dinitrate.

An acridine ring-substituted biacridinium derivative was prepared starting with N-[4-fluorophenyl]anthranilic acid (Pellón, supra). The acid was cyclized to 4-fluoro-9(10H)-acridone using polyphosphoric acid (Metz, supra). This acridone was alkylated with methyl 4-(bromomethyl)benzoate and the ester was saponified with methanolic sodium hydroxide. The resulting acid was dimerized with zinc and concentrated hydrochloric acid and the intermediate dimer was oxidized with dilute nitric acid to afford the difluorobiacridinium dinitrate. The NHS ester of this acid is obtained by coupling the acid with NHS in the presence of N,N′-dicyclohexyl-carbodiimide.

### a. Preparation of 10,10′-Bis[(4-carboxyphenyl)methyl]-3,3′-bis(trifluoromethyl)-9,9′-biacridinium dinitrate (Formula VI):

3-(Trifluoromethyl)-9(10H)-acridone. N-[3-(trifluoromethyl)phenyl]anthranilic acid (21.11 g, 0.075 mol), prepared as described by Pellón, was added in portions over 15 min to polyphosphoric acid (26.65 g) heated at 70-75° C. The reaction temperature was raised to 120° C. and maintained for 2 hours. The mixture was cooled to ˜80° C. and water (53.02 g) was added slowly to produce a solid. The mixture was stirred at ambient temperature overnight. The solid was collected and rinsed with water. The moist green solid (51.11 g) was boiled for 5 min with sodium carbonate (11.22 g, 0.106 mol) in water (150 mL). The solid was collected while hot and the filter cake was rinsed with water. The solid was partially dried with suction. The light green solid was dried further in a vacuum oven for 28 hours at 50° C. to afford a light yellow-green powder (18.83 g, 95%) that was an approximately equal mixture of 1- and 3-(trifluoromethyl)-9(10H)-acridones. A portion (3.00 g) of the solid was purified by flash chromatography on a silica gel column eluted with 60:40 hexanes:ethyl acetate to give 3-(trifluoromethyl)-9(10H)-acridone as a yellow solid (0.15 g).

10-[(4-Carboxyphenyl)methyl]-3-(trifluoromethyl)-9(10H)-acridone sodium salt. To a stirred suspension of sodium hydride (23 mg, 0.58 mmol) in tetrahydrofuran (4 mL) and dimethyl sulfoxide (1 mL) was added 3-(trifluoromethyl)-9(10H)-acridone (120 mg, 0.46 mmol) in portions over 5 min. The mixture was stirred at ambient temperature for 0.5 hours. To the resulting green solution was added methyl 4-(bromomethyl)benzoate (122 mg, 0.52 mmol). The mixture was heated for 4.25 hours at reflux and cooled to ambient temperature. The mixture was treated with acetic acid (2 drops) and stirred overnight. The solvent was concentrated on a rotary evaporator. The concentrate was diluted with water, ethyl acetate, and hexanes (6 mL each). The mixture was swirled and the resulting solid was collected by suction filtration. The filter cake was rinsed with water, dried briefly, and rinsed with hexanes. Additional solid that separated in the filtrate was collected as described for the first crop. The solids were dried and combined to give a light yellow powder (107 mg, 57%).

A portion of this powder (82 mg, 0.20 mmol) was suspended in methanol (0.80 g). The suspension was treated with an aqueous solution of 2.4% sodium hydroxide (0.67 g, 0.40 mmol). The mixture was heated at 65° C. for 1.5 hours, cooled to ambient temperature, and evaporated under a stream of nitrogen. Water (˜1 mL) was added and the mixture was acidified by the addition of a few drops of 37% hydrochloric acid. The resulting solid was collected by suction filtration. The filter cake was rinsed with water and dried to give a pale yellow powder (69 mg, 87%).

10,10′-Bis[(4-carboxyphenyl)methyl]-3,3′-bis(trifluoromethyl) 9,9′-biacridinium dinitrate (VI). To a stirred suspension of 10-[(4-carboxyphenyl)methyl]-3-(trifluoromethyl)-9(10H)-acridone (50 mg, 0.126 mmol) in acetone (0.80 g) was added zinc dust (142 mg, 2.15 mmol). The mixture was stirred for 5-10 min at ambient temperature. The mixture was treated with 37% hydrochloric acid (1.29 g, 13.1 mmol), cooled to 12 degrees C. and added over 1 hour and the mixture was stirred overnight. The bright yellow solid was collected by suction filtration and rinsed thoroughly with water. The filter cake was rinsed with acetone (a few drops), and dried briefly. The solid was treated with 6% nitric acid (2.18 g, 2.08 mmol) at 70° C. for 1.3 hours. Additional 6% nitric acid (1.09 g, 1.04 mmol) was added and heating continued for 1.4 hours. Upon cooling, the solid was collected by suction filtration and rinsed with a small amount of water. The filter cake was dried to afford a light orange powder (46 mg, 82%).

### b. Preparation of 10,10′-Bis[(4-carboxyphenyl)methyl]-2,2′-difluoro-9,9′-biacridinium dinitrate (Formula VII) and 10,10′-Bis[(4-N-succinimidyloxycarbonylphenyl)methyl]-2,2′-difluoro-9,9′-biacridinium dinitrate (Formula VIII):

N-(2-Fluorophenyl)anthranilic acid. A stirred suspension of copper powder (0.58 g, 0.009 mol) and potassium carbonate (8.46 g, 0.060 mol) in N,N-dimethylformamide was treated with 2-chlorobenzoic acid (19.17 g, 0.120 mol) and 4-fluoroaniline (27.10 g, 0.241 mol). The mixture was boiled for 2 hours. The cooled reaction mixture was poured into 300 mL of 1:1 water:37% hydrochloric acid. The solids were collected by suction filtration and washed with water. The purple solid was dissolved in sodium carbonate (11.47 g) in water (460 mL) and treated with activated carbon (11.48 g). The mixture was boiled for 5 min and filtered while hot. The cooled filtrate was acidified to pH 3 with 37% HCl. The resulting solid was collected by suction filtration and dried in a vacuum oven at 65° C. A light yellow powder (20.57 g, 74%) was obtained.

2-Fluoro-9(10H)-acridone. N-(2-fluorophenyl)anthranilic acid (19.75 g, 0.085 mol) was added in portions over 15 min to polyphosphoric acid (30.18 g) heated at ˜65° C. The reaction temperature was raised to 120° C. and maintained for 2 hours. The mixture was cooled to ˜80° C. and water (60 mL) was added slowly to produce a solid. The moist golden yellow solid was boiled for 5 min with sodium carbonate (12.62 g, 0.113 mol) in water (200 mL). The solid was collected while hot and the filter cake was rinsed with water. The solid was partially dried with suction. The solid was dried further in a vacuum oven at 60° C. to afford a light yellow-green powder (17.43 g, 96%).

10-[(4-Carboxyphenyl)methyl]-2-fluoro-9(10H)-acridone sodium salt. To a stirred suspension of sodium hydride (1.26 g, 31.5 mmol) in tetrahydrofuran (67 mL) and dimethyl sulfoxide (16 mL) was added 2-fluoro-9(10H)-acridone (5.33 g, 25.0 mmol) in portions over 10 min. The mixture was stirred at ambient temperature for 0.5 hours. To the resulting dark orange solution was added methyl 4-(bromomethyl)benzoate (6.44 g, 28.1 mmol). The mixture was heated for 2.25 hours at 70° C. and cooled to ambient temperature. The mixture was treated with acetic acid (0.75 g, 12.5 mmol) and stirred for 0.5 hours. The solvent was concentrated on a rotary evaporator. The concentrate was shaken with water and ethyl acetate (100 mL each). Hexanes (100 mL) was added, the mixture shaken to mix, and allowed to stand overnight. The resulting solid was collected by suction filtration. The filter cake was rinsed with water, dried briefly, and rinsed with hexanes. The cake was dried to give a light yellow powder (7.45 g, 82%). A portion of this solid (3.61 g, 9.99 mmol) was suspended in methanol (19 mL). The suspension was treated with a solution of sodium hydroxide (0.77 g, 18.7 mmol) in water (31 mL). The mixture was heated at reflux for 2 hours, cooled to ambient temperature, and placed in an ice-water bath. The resulting solid was collected by suction filtration and the filter cake was rinsed with cold water. The moist solid was mixed with acetone (50 mL), filtered, and dried in a vacuum oven for 1.5 hours at 50° C. to give a light yellow powder (3.47 g, 100%).

10,10′-Bis[(4-carboxyphenyl)methyl]-2,2′-difluoro-9,9′-biacridinium dinitrate (VII). To a stirred suspension of 10-[(4-carboxyphenyl)methyl]-2-fluoro-9(10H)-acridone sodium salt (2.08 g, 6.01 mmol) in acetone (50 mL) was added zinc dust (7.99 g, 121 mg atom). The mixture was stirred for 15 min at ambient temperature. The flask was cooled in a cold water bath and 37% hydrochloric acid (72.58 g, 737 mmol) was added dropwise over 3 hours, maintaining the reaction temperature at 20±2° C. during the addition. The zinc was consumed after stirring overnight. The bright yellow solid was collected by suction filtration and rinsed thoroughly with water. The filter cake was rinsed with acetone, ethyl acetate, and hexanes (2 mL each), and dried briefly. The solid was suspended in acetone (10 mL) and treated with 6% nitric acid (252.89 g, 241 mmol). The mixture was heated for 2 hours at 70° C., the color turning from yellow to olive to orange. Upon cooling, the solid was collected by suction filtration and rinsed with small amounts of water and 1:1 water:acetone. The solid was partially dried with suction and rinsed with ethyl acetate (50 mL) and hexanes. The solid was dried on a rotary evaporator at 45° C. for 1 hour to afford a light orange powder (1.65 g, 70%).

10,10′-Bis[(4-N-succinimidyloxycarbonylphenyl)methyl]-2,2′-difluoro-9,9′-biacridinium dinitrate (VIII). A solution of 10,10′-bis[(4-carboxyphenyl) methyl]-2,2′-difluoro-9,9′-biacridinium dinitrate (VII) (521 mg, 0.66 mmol) in N,N-dimethylformamide (16 mL) was treated with N-hydroxysuccinimide (232 mg, 2.02 mmol) and N,N′-dicyclohexylcarbodiimide (333 mg, 1.60 mmol). The mixture was stirred at ambient temperature for 24 hours. The reaction mixture was diluted with acetonitrile (8 mL) and stirred for 0.5 hours. The solids were removed by suction filtration and rinsed thoroughly with acetonitrile. The filtrate was concentrated on a rotary evaporator and the concentrate was added dropwise into rapidly stirred 75:25 ethyl acetate:hexanes (85 mL). The concentrate was rinsed in with 75:25 ethyl acetate:hexanes (15 mL). The resulting yellow solid was stirred for 0.5 hours and the solid was collected by suction filtration. The filter cake was rinsed with 75:25 ethyl acetate:hexanes (100 mL), followed by pure hexanes, and dried to give a yellow powder (553 mg, 85%).

Methoxy Substitution on Acridine Ring

An acridine ring-substituted biacridinium derivative was prepared starting with N-[4-methoxyphenyl]anthranilic acid (Pellón, supra). The acid was cyclized to 4-methoxy-9(10H)-acridone using polyphosphoric acid (Metz, supra). This acridone was alkylated with methyl 4-(bromomethyl)benzoate and the ester was saponified with methanolic sodium hydroxide. The resulting acid was dimerized with zinc and concentrated hydrochloric acid and the intermediate dimer was oxidized with dilute nitric acid to afford the dimethoxybiacridinium dinitrate. The NHS ester of this acid is obtained by coupling the acid with NHS in the presence of N,N′-dicyclohexylcarbodiimide.

### Preparation of 10,10′-Bis[(4-carboxyphenyl)methyl]-2,2′-dimethoxy-9,9′-biacridinium dinitrate (Formula IX) and 10,10′-Bis[(4-N-succinimidyloxycarbonylphenl)methyl]-2,2′-dimethoxy-9,9′-biacridinium dinitrate (Formula X):

2-Methoxy-9(10H)-acridone. N-(2-methoxyphenyl)anthranilic acid (19.70 g, 0.081 mol), prepared as described by Pellón, supra, was added in portions over 15 min to polyphosphoric acid (28.68 g) heated at ˜65° C. The reaction temperature was raised to 110-115° C. and maintained for 2 hours. The mixture was cooled to ˜80° C. and water (60 mL) was added slowly to produce a solid. The mixture was allowed to stand at ambient temperature overnight. The solid was crushed with a spatula, stirred for 10 min, collected by suction filtration, and rinsed with water. The moist golden yellow solid was boiled for 5 min with sodium carbonate (12.02 g, 0.113 mol) in water (150 mL). The solid was collected while hot and the filter cake was rinsed with water. The solid was partially dried with suction. The solid was dried further in a vacuum oven for 22 hours at 60° C. to afford a yellow powder (17.69 g, 97%).

10-[(4-Carboxyphenyl)methyl]-2-methoxy-9(10H)-acridone sodium salt. To a stirred suspension of sodium hydride (1.25 g, 31.3 mmol) in tetrahydrofuran (67 mL) and dimethyl sulfoxide (16 mL) was added 2-methoxy-9(10H)-acridone (5.63 g, 25.0 mmol) in portions over 10 min. The mixture was stirred at ambient temperature for 0.5 hours. To the resulting dark green solution was added methyl 4-(bromomethyl)benzoate (6.43 g, 27.5 mmol). The mixture was heated for 1.25 hours at 70° C. and cooled to ambient temperature. The mixture was treated with acetic acid (0.75 g, 12.5 mmol), stirred for 0.5 hours, and allowed to stand overnight. The solvent was concentrated on a rotary evaporator. The concentrate was shaken with water and ethyl acetate (100 mL each). Hexanes (100 mL) was added, the mixture shaken to mix, and allowed to stand overnight. The resulting solid was collected by suction filtration. The filter cake was rinsed with water, dried briefly, and rinsed with hexanes. The cake was dried to give a yellow, crystalline solid (7.29 g, 78%).

A portion of this solid (3.61 g, 9.66 mmol) was suspended in methanol (19 mL). The suspension was treated with a solution of sodium hydroxide (0.80 g, 19.4 mmol) in water (31 mL). The mixture was heated at reflux for 2 hours, cooled to ambient temperature, and placed. in an ice-water bath. The resulting solid was collected by suction filtration and the filter cake was rinsed with cold water. The moist solid was mixed with acetone, filtered, and dried to give a yellow powder (3.55 g, 96%).

10,10′-Bis[(4-carboxyphenyl)methyl]-2,2′-dimethoxy-9,9′-biacridinium dinitrate (IX). To a stirred suspension of 10-[(4-carboxyphenyl)methyl]-2-methoxy-9(10H)-acridone sodium salt (2.29 g, 6.00 mmol) in acetone (50 mL) was added zinc dust (7.92 g, 120 mg atom). The mixture was stirred for 25 min at ambient temperature. The flask was cooled in a cold water bath and 37% hydrochloric acid (72.94 g, 740 mmol) was added dropwise over 3 hours, maintaining the reaction temperature at 20±2° C. during the addition. The zinc was consumed after stirring overnight. The bright yellow solid was collected by suction filtration and rinsed thoroughly with water. The filter cake was rinsed with acetone, ethyl acetate, and hexanes (2 mL each), and dried briefly. The solid was suspended in acetone (5 mL) and treated with 6% nitric acid (158.39 g, 151 mmol). The mixture was heated for 0.5 hours at 40-45° C. and 0.5 hours at 55-60° C., the color turning from yellow to olive to vivid orange. Upon cooling, the solid was collected by suction filtration and rinsed with small amounts of water, 1:1 water:acetone, ethyl acetate, and hexanes. The solid was further dried in a vacuum oven at 45° C. for 4 hoursto afford a deep orange powder (1.69 g, 70%).

10,10′-Bis[(4-N-succinimidyloxycarbonylphenyl)methyl]-2,2′-dimethoxy-9,9′-biacridinium dinitrate (X). A solution of 10,10′-bis[(4-carboxyphenyl)methyl]-2,2′-dimethoxy-9,9′-biacridinium dinitrate (IX) (535 mg, 0.66 mmol) in N,N-dimethylformamide (16 mL) was treated with N-hydroxysuccinimide (232 mg, 2.02 mmol) and N,N′-dicyclohexylcarbodiimide (331 mg, 1.60 mmol). The mixture was stirred at ambient temperature for 24 hours. The reaction mixture was diluted with acetonitrile (8 mL) and stirred for 0.5 hours. The solids were removed by suction filtration and rinsed thoroughly with acetonitrile. The filtrate was concentrated on a rotary evaporator and the concentrate was added dropwise into rapidly stirred 75:25 ethyl acetate:hexanes (75 mL). The concentrate was rinsed in with 75:25 ethyl acetate:hexanes (25 mL). The resulting orange solid was stirred for 0.5 hours and the solid was collected by suction filtration. The filter cake was rinsed with 75:25 ethyl acetate:hexanes, followed by pure hexanes, and dried to give a deep orange powder (584 mg, 88%).

Asymmetric Substitution

### Preparation of 10-Methyl-10′-Toluic Acid Biacridinium Dinitrate Formula XIV) and its NHS ester (Formula XV)

10-Methyl-10′-Toluic Acid Biacridinium Dinitrate (XIV). The N-methyl-N′-toluic acid mixed biacridinium dinitrate (XIV) is synthesized by reductive coupling (Zn, HCl) of a mixture of N-methyl acridone (XI) and N-toluic acid acridone (XII). Use of a 4-5 equiv. excess of N-methyl acridone minimizes (though does not eliminate) formation of the unwanted bis-toluic acid product. The coupling chemistry gives a mixture of all three possible products; separation by analytical thin layer chromatography (TLC) clearly shows the bis-N-methyl product and the mono- and diacids. 1 gram of the N-toluic acid substituted acridone (XII) and 1.5 g of N-methylacridone (XI) are reductively coupled by treatment with zinc metal and concentrated HCl to give the biacridilidene (XIII) as a yellow solid. The solid is filtered and oxidized to the biacridiniun dinitrate using aqueous HNO₃ at reflux. Upon cooling, the biacridinium dinitrate precipitates from solution. The product is purified by crystallization and column chromatography. The 360 MHZ NMR spectrum of the mixed biacridinium dinitrate is consistent with the desired N-methyl-N′-toluic acid substituted biacridine structure (XIV); the NMR also shows some residual lucigenin in the sample.

The intermediate biacridylidene (XIII) mixture is oxidized in nitric acid and the product biacridinium dinitrate precipitates from solution on cooling. The isolated solid contains a large amount of lucigenin. Purification is accomplished by recrystallization and column chromatography.

10-Methyl-10′-Toluic Acid Biacridinium Dinitrate NHS Ester (XV). A solution of biacridinium dinitrate (XIV) (˜0.1 g), N-hydroxysuccinimide (˜0.1 g) and DCC (˜0.5 ml) in dimethylacetamide (5 ml) was stirred at room temperature overnight. The reaction was quenched with 2 ml of acetic acid, diluted with 50 ml of acetonitrile and filtered to remove precipitated DCU (dicyclohexylurea). Purification by column chromatography gave the final product in acceptable purity. The final product contains residual NHS and a trace of the bis-toluic acid biacridine according to NMR analysis. The N-hydroxysuccinimide (NHS) ester of the N-methyl-N-toluic acid biacridine has also been prepared using standard methods (NHS, dicyclohexylcarbodiimide [DCC] coupling). Proton NMR confirms the product as that in structure XV.

### Preparation of 10-[(4-carboxyphenyl)methyl]-10′-[(4-methoxy-carbonylphenyl)methyl]-9,9′-biacridinium dinitrate (Formula XVI) and 10-[(4-N-succinimidyloxycarbonylphenyl)methyl]-10′-[(4-methoxycarbonyl-phenyl)methyl]-9,9′-biacridinium dinitrate (Formula XVII):

A mixed dimerization procedure is utilized in the preparation of a mono-carboxymethyl ester biacridinium dinitrate (XVI). Coupling of 10-[(4-carboxyphenyl)methyl]-9(10H)-acridone sodium salt with an excess of 10-[(4-carbomethoxyphenyl)methyl]-9(10H)-acridone and subsequent oxidation with nitric acid gives a mixture of symmetrical and unsymmetrical biacridinium compounds. The desired asymmetrical biacridinium compound is isolated by flash chromatography. The free acid is coupled with NHS and DCC to give the mono-NHS ester (XVII).

10-[(4-carboxyphenyl)methyl]-10′-[(4-methoxycarbonylphenyl)methyl]-9,9′-biacridinium dinitrate (XVI). To a stirred suspension of 10-[(4-carboxyphenyl)methyl]-9(10H)-acridone sodium salt (0.84 g, 2.45 mmol) and 10-[(4-methoxycarbonylphenyl)methyl]-9(10H)-acridone (3.48 g, 9.90 mmol) in acetone (100 mL) was added zinc dust (13.05 g, 198 mmol). The mixture was stirred for 15 min at ambient temperature. The mixture was cooled to 15° C. in a cold water bath and 37% hydrochloric acid (122.94 g, 1248 mmol) was added dropwise over 4.2 hours, maintaining the reaction temperature at 20±2° C. during the addition. The mixture was stirred overnight at ambient temperature. The bright yellow solid was collected by suction filtration and rinsed thoroughly with water. The filter cake was rinsed with acetone, ethyl acetate, and hexanes (5 mL each), and dried to afford a yellow powder (3.89 g). A portion (2.00 g) of this solid was treated with 6% nitric acid (160.97 g, 153.3 mmol). The mixture was heated for 2 hours at 70° C., the color turning from yellow to light orange. Upon cooling to 15° C. for 0.5 hours, the solid was collected by suction filtration, rinsed with water, and dried. The solid was further dried on a rotary evaporator at 50° C. for 1 hour to give a light orange powder (2.19 g). A portion of the solid was purified by flash chromatography on a silica gel column eluted with 85:15 acetonitrile:0.1 N nitric acid. The fractions containing the half-ester were evaporated to give a moist solid (0.27 g). This solid was combined with 6% nitric acid (13.51 g) and heated for 1.5 hours at 70° C. The solid was collected by suction filtration, rinsed with a small amount of water, and dried. The product was obtained as an orange powder (111 mg).

10-[4-(N-succinimidyloxycarbonylphenyl)methyl]-10′-[4-(methoxy-carbonylphenyl)methyl]-9,9′-biacridinium dinitrate (XVII). A solution of 10-[4-(carboxyl)phenylmethyl]-10′-[4-(methoxycarbonyl)phenylmethyl]-9,9′-biacridinium dinitrate (XVI) (99 mg, 0.13 mmol) in N,N-dimethylformamide (1.94 g) was treated with N-hydroxysuccinimide (21 mg, 0.18 mmol) and N,N′-dicyclohexylcarbodiimide (32 mg, 0.16 mmol). The mixture was stirred at ambient temperature for 16.5 h. To the mixture was added NHS (21 mg, 0.18 mmol) and stirring continued for 6 h. Another portion of NHS (21 mg, 0.18 mmol) and DCC (30 mg, 0.15 mmol) was added and stirring was continued for 27.5 h. Another portion of DCC (9 mg, 0.04 mmol) was added and stirring was continued for 4 h. The reaction mixture was diluted with acetonitrile (2 mL) and stirred for 1 h. The mixture was further diluted with acetonitrile (10 mL) and the solids were removed by suction filtration and rinsed thoroughly with acetonitrile. The filtrate was concentrated on a rotary evaporator and the concentrate was poured slowly into rapidly stirred ethyl acetate (100 mL). The resulting yellow solid was stirred overnight. The solid was collected by suction filtration. The filter cake was rinsed with ethyl acetate and hexanes and dried to give a yellow powder (250 mg).

Another simplified version of synthesis for assymetric compounds according to the present invention is performed by the use of different acridone reagents (e.g., an acridone with an A substituent in the 10 position and an acridone with a B substituent in the 10 position which are reacted in bulk (e.g., a ‘single pot’ reaction as above) so that a mixture of 10,10′-AA-biacridine, 10,10′-BB-biacridine, and 10.10′AB-biacridine is provided. The batch reaction product may or may not then be separated into one, two or three of the biacridines, as by HPLC or other separation methods, depending upon the physical and chemical property differences amongst the three reaction products.

3. Analysis of Biacridinium Dinitrate Acids for Light Output

Materials for Analysis of Biacridinium Dinitrate Labels

Phosphoric Acid; and ammonium chloride (Baker Chemical Co. Phillipsburg, Pa.), Sodium phosphate, dibasic, anhydrous; glycine; sodium hydroxide; and Tris(hydroxymethyl) aminomethane hydrochloride (Tris Hcl); maltose and potassium superoxide (Sigma, St. Louis, Mo.), N,N-Dimethylformamide (99.9+%); and 2-methyl-2-propanol (Aldrich, Milwaukee, Wis.) BupH™ Modified Dulbecco's PBS Packs (0.008 M sodium phosphate, 0.002 M potassium phosphate, 0.14 M sodium chloride and 0.01 M potassium chloride, pH 7.4); SuperBlock® Blocking Buffer in PBS (protein containing blocking solution in 0.01 M sodium phosphate, 150 mM sodium chloride and Kathong® an anti-microbial agent at pH 7.4) (Kathon is a registered trademark of Rohm and Haas), sequanal grade dimethylformamide (Dry DMF); Biotinylated Bovine Serum Albumin Coated White Microwell plate; Tween® 20 nonionic detergent Tween 20 is a registered trademark of ICI Americas); Streptavidin; Slide-A-Lyzer® Dialysis Cassette (U.S. Pat. No. 5,503,741); 18-gauge needle and 5 ml syringe (Pierce Chemical Company, Rockford, Ill.).

Equipment

High Performance Liquid Chromatography (HPLC) System Hewlett-Packard Series 1100 Chem Station (Hewlett-Packard)

Gel Filtration Standards, HPLC Gel Filtration Column-Bio-Sil Sec-250 (300 mm×7.8 mm); HPLC Guard Column-Bio-Sil Sec-250 (80 mm×7.8 mm) (BioRad, Hercules, Calif.)

MLX Microtiter® Plate Luminometer (Dymex Technologies, Inc Chantily, Va.)

Biacridinium dinitrate acids were prepared in dry DMF and diluted in SuperBlock Blocking Buffer in PBS. The samples were added to a microwell plate at 10 ug/well. The plate was placed on the Dynex MLX Microtiter® Plate Luminometer. The trigger solution was injected into the wells of the plate at 100 ul/well. The wells were read 2 seconds/well to determine total relative light units (RLU).

Analysis of Biacridinium Dinitrate Acids that are Symmetrically Substituted on the Toluic Acid for Light Output

Symmetrical Substitutions on Toluic Acid

| Biacridinium Dinitrate Acid | Net Relative Light |
|---|---|
| Substitution on Toluic Acid | Units at 10 μg/well |
| 10,10′-ρ-Toluic acid-9,9′- | 610,669 |
| Biacridinium dinitrate (XVIII) | |
| 10,10′-Bis[(4-carboxy-2- | 1,039,755 |
| fluorophenyl)methyl]-9,9′- | |
| Biacridinium dinitrate (II) | |
| 10′-Bis[(4-carboxy-2- | 290,596 |
| methoxyphenyl)methyl]-9,9′- | |
| Biacridinium dinitrate (IV) | |

Analysis of Biacridinium Dinitrate Acids that are Asymmetrically Substituted on the Acridine Nucleus

Asymmetric Substitution on the N Position
of Biacridinium Dinitrate

| | Net Relative Light |
|---|---|
| Biacridinium Dinitrate | Units at 10 μg/well |
| N-Methyl-N′-Toluic acid Biacridinium | 527,656 |
| dinitrate (XIV) | |

Analysis of Biacridinium Dinitrate Acids that are Symmetrically Substituted on the Acridine Ring for Light Output

Acridine ring-substituted biacridinium salts were tested similarly to the other biacridines on a Laboratory Technologies ACCULYTE 10 Series Luminescence Counter. The sample was prepared in dry DMF and diluted in SuperBlock® Blocking Buffer in PBS. The biacridinium salts were added to a test tube at 10 pg/tube. The trigger solution was injected into the test tube 200 ul/tube. The tube was read at 2 seconds/tube to determine total RLU.

Symmetric Substitution on the Acridine Ring

| | Relative Light |
|---|---|
| Acridine Ring Substituted Biacridinium Dinitrate | Units at 10 pg/tube |
| 10,10′-Bis [(4-carboxyphenyl)methyl]-2,2′- | 579,078 |
| dimethoxy-9,9′-Biacridinium dinitrate (IX) | |
| 10,10′-Bis[4-carboxyphenyl)methyl]-2,2′- | 481,611 |
| difluoro-9,9′-Biacridinium dinitrate (VII) | |
| 10,10′-Bis[(4-carboxyphenyl)methyl]-3,3′- | 71,683 |
| bis(trifluoromethyl)-9,9′-Biacridinium dinitrate(VI) | |

4. Preparation of Biacridinium Conjugates

Streptavidin was dialyzed into 0.1 M Phosphate at pH 8.5 for a final concentration of 4 mg/ml. The biacridinium compounds were prepared in dry DMF and added to the streptavidin for a final 16:1 molar excess of biacridine to streptavidin. The N-hydroxysuccinimide reaction was allowed to react for 1 hour at room temperature. The reaction was stopped with glycine. To purify the conjugates, they were dialyzed in a Slide-a-Lyzer® Cassette into 0.1 M Tris HCl pH 6.0 (1 ml of conjugate/500 ml buffer) with three buffer changes. The streptavidin conjugates had two spectrophotometric peaks using a Hitachi spectrophotometer at both 370 nm (biacridinium label) and 280 nm (streptavidin).

5. Analysis by HPLC of Biacridinium Conjugates

HPLC Analysis of the conjugates was run with Dulbecco's Modified PBS pH 6.0 containing 10% DMF running buffer on a Gel Filtration Column (Bio-Sil® Sec-250 300 mm×7.8 mm) in sequence.

Substitution on the Toluic Acid

| | | Retention | | Retention | |
|---|---|---|---|---|---|
| | | Time at | Height | Time | Height |
| HPLC Standard | MW | A280 | (mAu) | A370 | (mAu) |
| Thyroglobulin | 670,000 | 7.115 | 63.20991 | | |
| Bovine gamma globulin | 158,000 | 9.459 | 13.27857 | | |
| Chicken ovalbumin | 44,000 | 10.645 | 82.55029 | | |
| Equine myoglobin | 17,000 | 12.579 | 75.4627 | 12.582 | 123.8666 |
| Cyanocobalamin | 1,350 | 13.503 | 98.36756 | 13.503 | 163.5457 |
| | | | 138.795 | | |
| 10,10′-para-Toluic acid- | | 10.019 | 3.11764 | 10.035 | 3.68414 |
| 9,9′-Biacridinium | | | | | |
| dinitrate labeled | | | | | |
| Streptavidin | | | | | |
| | | 11.099 | 10.11713 | 11.093 | 12.56361 |
| 10,10′-Bis[(4- | | 10.006 | 3.05856 | 10.029 | 25.6642 |
| carboxy-2-fluoro- | | | | | |
| phenyl]methyl]- | | | | | |
| 9,9′-Biacridinium | | | | | |
| dinitrate labeled | | | | | |
| Streptavidin | | | | | |
| | | 11.073 | 11.05375 | 11.06 | 74.3358 |
| 10,10′-Bis(4- | | 9.938 | 2.63377 | 9.926 | 2.88410 |
| carboxy-2-methoxy- | | | | | |
| phenyl)methyl]- | | | | | |
| 9,9′-biacridinium | | | | | |
| dinitrate labeled | | | | | |
| streptavidin | | | | | |
| | | 10.978 | 14.36486 | 10.97 | 16.45686 |

Substitution on the Acridine Ring

| | | Retention | | Retention | |
|---|---|---|---|---|---|
| HPLC | | Time at | Height | Time | Height |
| Standard | MW | A280 | (mAu) | A370 | (mAu) |
| Thyroglobulin | 670,000 | 6.994 | 92.46 | | |
| Bovine gamma globulin | 158,000 | 9.37 | 141.29 | | |
| Chicken ovalbumin | 44,000 | 10.551 | 137.73 | | |
| Equine myoglobin | 17,000 | 12.453 | 221.33 | | |
| Cyanocobalamin | 1,350 | 13.339 | 421.14 | | |
| 10,10′-para-Toluic acid- | | 11.023 | 14.90 | 11.013 | 12.10 |
| 9,9′-Biacridinium | | | | | |
| dinitrate labeled | | | | | |
| streptavidin | | | | | |
| | | 9.952 | 4.22 | 9.948 | 3.47 |
| 10,10′-Bis[(4- | | 10.872 | 59.93 | 10.862 | 9.10 |
| carboxyphenyl-methyl- | | | | | |
| 2,2′-dimethoxy- | | | | | |
| 9,9′-Biacridinium | | | | | |
| dinitrate labeled | | | | | |
| streptavidin | | | | | |
| 10,10′-Bis(4- | | 11.021 | 8.67 | 11.022 | 5.39 |
| carboxyphenyl)-methyl]- | | | | | |
| 2,2′-difluoro- | | | | | |
| 9,9′-Biacridinium | | | | | |
| dinitrate labeled | | | | | |
| streptavidin | | | | | |
| Asymmetric Run At | | | | | |
| The Same Time | | | | | |
| 10-[(4-carboxyphenyl) | | 11.383 | 63.33441 | 11.435 | 27.69574 |
| methyl]-10′-[(4- | | | | | |
| methoxycarbonyl- | | | | | |
| phenyl)-methyl]- | | | | | |
| 9,9′-Biacridinium | | | | | |
| dinitrate labeled | | | | | |
| streptavidin | | | | | |
| | | 10.286 | 8.08736 | 10.305 | 3.76629 |

Asymmetric Biacridinium Dinitrate

| | | Retention | | Retention | |
|---|---|---|---|---|---|
| HPLC | | Time at | Height | Time | Height |
| Standard | MW | A280 | (mAu) | A370 | (mAu) |
| Thyroglobulin | 670,000 | 10.531 | 124.693 | | |
| Bovine gamma globulin | 158,000 | 13.909 | 153.384 | | |
| Chicken ovalbumin | 44,000 | 15.64 | 182.550 | | |
| Equine myoglobin | 17,000 | 18.447 | 223.585 | 18.451 | 276.793 |
| Cyanocobalamin | 1,350 | 19.865 | 345.100 | 19.865 | 447.105 |
| Streptavidin | | 14.719 | 2.78770 | | |
| | | 16.473 | 54.55226 | | |
| N-Methyl-N-Toluic | | 14.965 | 11.6549 | 14.977 | 9.37322 |
| Biacridinium | | | | | |
| dinitrate labeled | | | | | |
| streptavidin | | | | | |
| | | 16.568 | 72.85978 | 16.585 | 61.92606 |

6. Analysis by Functional Assay of Biacridinium Conjugates

Conjugates were prepared in SuperBlock® Blocking Buffer in PBS containing 0.05% Tween® 20 at 1 mg/ml. Samples were added to a biotinylated BSA coated white microwell plate and incubated for 1 hour at room temperature on a shaker platform. The wells of the microwell plate were washed with PBS containing 0.05% Tween-20. The plate was placed on a MLX Microtiter® Plate Luminometer set to inject 100 ul of the Trigger Solution/well. The wells were read 2 seconds/well to determine total integral RLU.

Functional Assay of Biacridinium-Streptavidin Conjugates Uniformly
Symmetrically Substituted on the Toluic Acid Ring

| Biacridinium Conjugated to | Net Relative Light |
|---|---|
| Streptavidin | Units at 1 μg/ml |
| 10,10′-ρ-Toluic acid-9,9′- | 61,298 |
| Biacridinium dinitrate (XVIII) | |
| 10,10′-Bis[(4-carboxy-2- | 58,432 |
| fluorophenyl)methyl]-9,9′- | |
| Biacridinium dinitrate (II) | |
| 10,10′-Bis[(4-carboxy-2- | 61,006 |
| methoxyphenyl)methyl]-9,9′- | |
| Biacridinium dinitrate (IV) | |

Functional Assay of Biacridinium-Streptavidin Conjugates Uniformly
Symmetrically Substituted on the Biacridine Ring

| Biacridinium Compound Conjugated | Net Relative Light |
|---|---|
| to Streptavidin | Units at 1 μg/ml |
| 10,10′-ρ-Toluic acid-9,9′- | 22,932 |
| Biacridinium dinitrate (XVIII) | |
| 10,10′-Bis[(4-carboxyphenyl)methyl]- | 2,194 |
| 2,2′-dimethoxy-9,9′-Biacridinium | |
| dinitrate (IX) | |
| 10,10′-Bis[(4-carboxyphenyl)methyl]- | 17,413 |
| 2,2′-difluoro-9,9′-Biacridinium | |
| dinitrate (VII) | |

Functional Assay of Biacridinium-Streptavidin Conjugates
Using Asymmetrical Biacridinium Dinitrate

| Biacridinium Dinitrate Compound | Net Relative Light |
|---|---|
| Conjugated to Streptavidin | Units at 1 μg/ml |
| N-Methyl-N′-Toluic Biacridinium | 34,630 |
| dinitrate (XIV) | |
| 10-[(4-Carboxyphenyl)methyl]-10′- | 18,143 |
| [(4-methoxycarbonylphenyl)methyl]- | |
| 9,9′-Biacridinium dinitrate labeled | |
| streptavidin (XVI) | |

7. Measurement of Multiple Luminescent Molecules in a Single Sample Using at Least One Signal Reagent

In this example it is demonstrated that at least one luminescent molecule can be triggered with at least one signal and that at least one other luminescent molecule can be triggered by at least one other signal, all in the same sample, and that light emitted from these luminescent molecules can be differentiated, detected and measured (a Berthold Lumat LB 9501 luminometer was used in this example). Table 1 shows the relative total counts for various luminescent molecules when 5 microliters of each solution of the molecules are triggered simultaneously with the signal reagent of this example. Unique kinetic curves are generated by triggering of 5 microliters of chlorin e₆ solution (Porphyrin Products. Logan, Utah) and 1 microliter of 10,10′-p-toluic acid-9,9-biacridine together in the same test tube with a signal reagent of the invention (signal reagent 1: i.e. SR 1) containing 1.0 mg d-L-fructose, 0.13 ml 2-methyl-2-propanol and 440 mM KO₂ per mole of a 0.02 M aqueous solution of sodium tetraborate. The differences in kinetics were noted for each luminescent molecule as were the two different light peaks when both molecules were flashed in the same sample with a signal reagent of the invention. Four distinct luminescent molecules are further used in this example. Eight microliters of 4-(2-succinimidyloxycarbonylethyl)phenyl-10-methylacridinium-9-carboxylate trifluoromethane sulfonate-labeled DNA probe for the detection of Neisseria gonorrhoeae in 0.01 M lithium succinate (pH 5) and 0.1% (w/v) lithium lauryl sulfate (Gen Probe, Inc., San Diego, Calif.), 15 microliters of the 10,10′-acetic acid-9,9-biacridine-N-hydroxysuccinimide derivative in 0.01 M NaPhosphate, 5 microliters of deuteroporphyrin 1×2 HCl (Porphyrin Products, Logan, Utah) in acetonitrile (Aldrich) and 5 microliters of hemin in water are contacted firstly with 200 microliters of a combination of 1.5% H₂O₂+0.1 N HNO₃ (signal reagent 2: i.e., SR 2) and, 0.5 seconds later, with 200 microliters of 1 N NaOH (signal reagent 3: i.e., SR 3). The kinetic curves generated by each of these four luminescent molecules when separately triggered with SR 2+SR 3 were again recorded. Kinetic curves were generated by the SR 2+SR 3 separate triggering of 8 microliters of the acridinium ester conjugate and 15 microliters of the biacridine-NHS derivative, and then the simultaneous triggering of these two molecules of the example in the same sample. Following the triggering of these four luminescent molecules in the same sample with SR 1+SR 2, they were then separately triggered with 200 ul of a signal reagent containing 28 microliters of a 1% solution (w/v) of sodium di-2-ethylhexyl sulfosuccinate (AOT: K&K Laboratories, Cleveland, Ohio), 3.4 mg dextrose, 14 microliters of a 5-amino-2,3-dihydro-1,4-phthalazinedione (luminol) solution (880 microliters of 10 mM luminol in H₂O added to 100 ml of 0.1 M Trizma base in H₂O), and KO₂ to bring the pH to 12.86 per 1.0 ml of 0.1 M. SR 4. Kinetic curves were generated by these four luminescent molecules together in the same sample when triggered with SR 2+SR 3 and then subsequently with SR 4. Luminol is the non-limiting luminescent reactant of this example, however, and many other luminescent reactants such as enzymes, dioxetanes, peroxyoxylates, isoluminols, ethylbenzene, hypoxanthines and isoxanthopterins can be utilized. In this example, light emitted from luminescent molecules triggered with the first signal reagent is differentiated, detected and measured and then, following triggering of the other luminescent molecules, that separate light emitted is differentiated, detected and measured (a Berthold Lumat LB 9501 luminometer was used in this example). Among the ways differentiation can be accomplished are through the use of differences in the kinetics of light output produced by each luminescent molecule and differences in the wavelengths of light produced by each luminescent molecule (e.g., the acridinium ester derivative of the example emits light at a peak wavelength of 435 lambda while the biacridine derivative of the example emits light at a peak wavelength of 495 lambda). In this example, the acridinium ester derivative molecules and biacridine derivative molecules are triggered with a first signal reagent and the tetrapyrrole molecules are separately triggered with a second signal reagent, all in the same tube. When these luminescent molecules are used as labels to measure analytes of interest, the amount of light differentiated, detected and measured is directly or indirectly proportional to the amounts of specific analytes of interest depending on the assay format and conditions.

TABLE 1
Relative Light Productions Of Various Luminescent
Molecules When Triggered With Signal Reagents
Of Example 5 (Counts/5 sec./100)

| Molecules | SR 2 and 3 | SR 4 | SR 1 |
|---|---|---|---|
| A.D.1 | 1.7 | 766 | 1816 |
| A.D.2 | O.6 | 10500 | 5 |
| B.D.1 | 5.1 | 2612 | 28 |
| B.D.2 | 453.3 | 88656 | 62843 |
| DPIX | 7.7 | 78 | 16 |
| Hemin | 0.6 | 32694 | 47 |
| Zero | 0.5 | 83 | 2 |

1. A.D.1: Dimethylacridinium ester-labeled antibody to TSH Ciba Corning Diagnostics, Walpole, MA)
2. A.D.2: 4-(2-succinimidyloxycarbonylethyl)phenyl-10-methylacridinium 9-carboxylate trifluoromethane sulfonate-labeled DNA probe (Gen Probe, Inc., San Diego, CA)
3. B.D.1: 10,10′-Acetic acid-9,9′-Biacridinium-NHS derivative
4. B.D.2: 10,10′-p-Toluic acid-9,9′-Biacridinium-BSA conjugate
5. DPIX: Deuteroporphyrin IX•2HCl

What is claimed is:

1. A composition comprising a 10,10′-substituted-9,9′-biacridene conjugated to antigen, antibody, macromolecule, protein, nucleic acid, polymer, analyte, binding partner of analyte, ligand of binding partner to analyte, hapten conjugate or hapten wherein the substituent substituted at the 10 position is different from the substituent substituted at the 10′ position.

2. A composition comprising a 10,10′-substituted-9,9′-biacridene conjugated to antigen, antibody, macromolecule, protein, nucleic acid, polymer, analyte, binding partner of analyte, ligand of binding partner to analyte, hapten conjugate or hapten wherein the substituent substituted at the 10 and 10′ positions are the same and substituents substituted elsewhere on the biacridine ring are different from the substituents at the 10 and 10′ positions.

3. A composition comprising a 10,10′-substituted-9,9′-biacridene conjugated to antigen, antibody, macromolecule, protein, nucleic acid, polymer, analyte, binding partner of analyte, ligand of binding partner to analyte, hapten conjugate or hapten wherein the substituent substituted at the 10 and 10′ positions are the same and substituents substituted elsewhere on the biacridine ring are identically substituted on the biacridine rings.

4. A composition comprising a symmetric, uniformly symmetric or asymmetric 10,10′-substituted-9,9′-biacridene conjugated to antigen, antibody, macromolecule, protein, nucleic acid, polymer, analyte, binding partner of analyte, ligand of binding partner to analyte, hapten conjugate or hapten wherein at least one substituent on the biacridene is a reactive group.

5. The composition of claim 4 wherein said at least one of said biacridine reactive group substituent is linked to said 9,9′-biacridine having an N-hydroxysuccinimide group, sulfo-N-hydroxysuccinimide, polyfluorophenol or imidazole activated ester.

6. The composition of claim 4 wherein said at least one of said 10-substituent and said 10′-substituent is linked to a succinimidyloxycarbonyl group, sulfo-N-hydroxysuccinimide, polyfluorophenol or imidazole activated ester.

7. An asymmetric 10,10′-substituted-9,9′-biacridine wherein a substituent substituted at the 10 position is different from a substituent substituted at the 10′ position of the 9,9′-biacridine.

8. The asymmetric 10,10′-substituted-9,9′-biacridine of claim 7 wherein said 10-substituent and/or said 10′-substituent is derivatized with N-hydroxysuccinimide group, polyfluorophenol group, imidazole activated ester, succinimidyloxycarbonyl group and/or sulfo-N-hydroxysuccinimide group.

9. An asymmetric 10,10′-substituted-9,9′-biacridine compound wherein a fused phenyl ring on said 9,9′-biacridine is differently substituted with group or groups other than hydrogen.

10. A symmetric 10,10′-substituted-9,9′-biacridine compound wherein a fused phenyl ring on said 9,9′-biacridine is substituted with a group or groups differing from the group substituted at the 10 and 10′ positions.

11. A uniformly symmetric 10,10′-substituted-9,9′-biacridine compound wherein fused phenyl rings on said 9,9′-biacridine are identically substituted with group or groups other than hydrogen.

12. The compound of claim 9 wherein a substituent present on said fused phenyl ring on said 9,9′-biacridine is selected from the group consisting of for example alkyl groups, alkenyl groups, halogenated alkyl, sulfonate, alkoxy, aryloxy, nitrile, inorganic acid groups, hetero atoms, perfluoroalkyl group, aryl groups, amino, carboxyl, hydroxyl and halogen.

13. The compound of claim 10 wherein a substituent present on said fused phenyl ring on said 9,9′-biacridine is selected from the group consisting of for example alkyl groups, alkenyl groups, halogenated alkyl, sulfonate, alkoxy, aryloxy, nitrile, inorganic acid groups, hetero atoms, perfluoroalkyl group, aryl groups, amino, carboxyl, hydroxyl and halogen.

14. The compound of claim 11 wherein a substituent present on said at least one fused phenyl ring on said 9,9′-biacridine is selected from the group consisting of for example alkyl groups, alkenyl groups, halogenated alkyl, sulfonate, alkoxy, aryloxy, nitrile, inorganic acid groups, hetero atoms, perfluoroalkyl group, aryl groups, amino, carboxyl, hydroxyl and halogen.

15. The compound of claim 10 which is symmetric and at least one ring on each of the two acridine moieties forming the 9,9′-biacridine is substituted with a different group or groups as found at the 10 and 10′ positions.

16. The compound of claim 11 which is uniformly symmetrical and at least one ring on each of the two acridine moieties forming the 9,9′-biacridine is identically substituted with the same group or groups.

17. The compound of claim 10 wherein said 10,10′-substituted-9,9′-biacridine is a 10,10′-para-toluic acid-9,9′-biacridine, a 10,10′-para-toluo-9,9′-biacridine, a 10,10′-aceto-9,9′-biacridine or a 10,10′-acetic acid-9,9′-biacridine.

18. The compound of claim 11 wherein said 10,10′-substituted-9,9′-biacridine is a 10,10′-para-toluic acid-9,9′-biacridine, a 10,10′-para-toluo-9,9′-biacridine, a 10,10′-aceto-9,9′-biacridine or a 10,10′-acetic acid-9,9′-biacridine.

19. The symmetric 10,10′-substituted-9,9′-biacridine compound of claim 10 wherein each acridine has at least one substituent other than hydrogen attached to rings thereof.

20. A chemiluminescent system for emitting measurable light useful in chemical assay, immunoassay, ligand binding assay, protein assay, particle assay, hapten assay, macromolecule assay or nucleotide assay, said system comprising: asymmetric 10,10′-substituted-9,9′-biacridine compound of claim 1 having an energy of activation, signal or combination of signals capable of overcoming the energy of activation of asymmetric 10,10′-substituted-9,9′-biacridine, said asymmetric 10,10′-substituted-9,9′-biacridine being bound to analyte, binding partner of analyte and/or ligand of binding partner to analyte.

21. A chemiluminescent system for emitting measurable light useful in chemical assay, immunoassay, ligand binding assay, protein assay, particle assay, hapten assay, macromolecule assay or nucleotide assay, said system comprising: symmetric 10,10′-substituted-9,9′-biacridine compound of claim 2 having an oxidation potential, signal or combination of signals capable of overcoming the oxidation potential of the symmetric 10,10′-substituted-9,9′-biacridine, said symmetric 10′-substituted-9,9′-biacridine being bound to analyte, binding partner of analyte and/or ligand of binding partner to analyte.

22. A chemiluminescent system for emitting measurable light useful in chemical assay, immunoassay, ligand binding assay, protein assay, particle assay, hapten assay, macromolecule assay or nucleotide assay, said system comprising: uniformly symmetric 10,10′-substituted-9,9′-biacridine compound of claim 3 having an oxidation potential, signal or combination of signals capable of overcoming the oxidation potential of the uniformly symmetric 10,10′-substituted-9,9′-biacridine, said uniformly symmetric 10,10′-substituted-9,9′-biacridine being bound to analyte, binding partner of analyte and/or to ligand of binding partner to analyte.

23. A chemiluminescent system comprising the symmetric, uniformly symmetric or asymmetric 10,10′-substituted-9,9′-biacridine of claim 4 having signal or signals with oxidant or oxidants, chelating agent, sulfoxide, reducing sugar, alcohol., singlet oxygen, singlet oxygen generators, light, heat, energy, radiofrequency discharge, electromagnetic energy, electricity, microwave, ultrasound, chemical, singlet oxygen emitting molecules or a combination of signals capable of overcoming the energy of activation of the 10,10′-substituted-9,9′-biacridine.

24. The chemiluminescent system of claim 4 wherein said analyte is nucleic acid, antigen, antibody, hapten, hapten conjugate, macromolecule, protein and/or polymer.

25. The chemiluminescent system of claim 23 wherein said 10,10′-substituted-9,9′-biacridine is bound to analyte, binding partner of analyte or to ligand of binding partner of analyte by means of biotin-avidin or biotin-streptavidin bridge.

26. The chemiluminescent system of claim 31 wherein oxidant is potassium superoxide, buffer solution comprises aqueous sodium tetraborate, chelating agent comprises EDTA, sulfoxide comprises DMSO, reducing sugar comprises D(-) fructose and the system further comprises alcohol 2-methyl-2-propanol.

27. A method for using the symmetric, uniformly symmetric or asymmetric 10,10′-substituted-9,9′-biacridine of claim 4 in luminescent homogeneous assay for detecting the presence of or measuring the amount of analyte in sample(s) comprising:
(a) providing a solid phase coated with a specific binding partner for said analyte;
(b) contacting said solid phase with said sample and with a predetermined amount of said symmetric, uniformly symmetric or asymmetric 10,10′-substituted-9,9′-biacridine-analyte conjugate or biacridine-analyte binding partner conjugate, said symmetric uniformly symmetric or asymmetric 10,10′-substituted-9,9′-biacridine having energy of activation, and with predetermined amount of signal generator conjugated to binding partner to analyte or conjugated to binding partner to analyte preventing unbound symmetric uniformly symmetric or asymmetric 10,10′-substituted-9,9′-biacridine-analyte conjugate from mediating luminescence, at least some of said binding partner binding to at least some of said symmetric uniformly symmetric or asymmetric 10,10′-substituted-9,9′-biacridine-analyte conjugate or biacridine-analyte binding partner conjugate.
(c) contacting the solid phase from (b) with signal or signals that overcome the energy of activation of the symmetric uniformly symmetric or asymmetric 10,10′-substituted-9,9′-biacridine in the bound symmetric uniformly symmetric or asymmetric 10,10′-substituted-9,9′-biacridine analyte conjugate to emit light; and
(d) measuring the amount of light emitted in (c) wherein said amount of emitted light will be indirectly proportional to the amount of analyte present in said sample when using biacridine-analyte conjugate in a competitive assay for hapten molecules and directly proportional to the amount of emitted light when using biacridine-analyte binding partner conjugate in a sandwich assay for macromolecules.

28. A method for using the symmetric, uniformly symmetric or asymmetric 10,10′-substituted-9,9′-biacridine of claim 4 in luminescent homogeneous assays for detecting the presence of or measuring the amount of analyte in sample(s) comprising:
(a) contacting said sample and with a predetermined amount of said symmetric, uniformly symmetric or asymmetric 10,10′-substituted-9,9′-biacridine-analyte conjugate or biacridine-analyte binding partner conjugate, said symmetric uniformly symmetric or asymmetric 10,10′-substituted-9,9′-biacridine having energy of activation, and with predetermined amount of signal generator conjugated to analyte or conjugated to binding partner to analyte preventing unbound symmetric uniformly symmetric or asymmetric 10,10′-substituted-9,9′-biacridine-analyte conjugate from mediating luminescence, at least some of said binding partner binding to at least some of said symmetric uniformly symmetric or asymmetric 10,10′-substituted-9,9′-biacridine-analyte conjugate or biacridine-analyte binding partner conjugate.
(b) contacting the sample from (a) with signal or signals that overcome the energy of activation of the symmetric uniformly symmetric or asymmetric 10,10′-substituted-9,9′-biacridine in the bound symmetric uniformly symmetric or asymmetric 10,10′-substituted-9,9′-biacridine analyte conjugate or biacridine-analyte binding partner conjugate to emit light; and
(c) measuring the amount of light emitted in (c) wherein said amount of emitted light will be indirectly proportional to the amount of analyte present in said sample when using biacridine-analyte conjugate in a competitive assay for hapten molecules and directly proportional to the amount of emitted light when using biacridine-analyte binding partner conjugate in a sandwich assay for macromolecules.

29. A method for using symmetric, uniformly symmetric or asymmetric 10,10′-substituted-9,9′-biacridine of claim 4 in a chemiluminescent heterogeneous assay for detecting the presence of multiple analytes in a sample comprising:
(a) providing a solid phase coated with specific binding partner for each analyte said binding partner being specific for said each analyte
(b) contacting said solid phase with said sample and with non-biacridine label-analyte conjugate and symmetric, uniformly symmetric or asymmetric 10,10′-substituted-9,9′-biacridine label-analyte conjugate, at least some of said non-biacridine label-analyte conjugate binding specifically to at least some of said non-biacridine label-analyte conjugate solid phase binding partner and at least some of said biacridine label-analyte conjugate binding specifically to at least some of said symmetric, uniformly symmetric or asymmetric 10,10′-substituted-9,9′-biacridine label-analyte conjugate solid phase binding partner;
(c) separating unbound conjugates from bound conjugates by washing said contacted solid phase;
(d) contacting said washed solid phase in (c) with signal or signals specific for said symmetric, uniformly symmetric or asymmetric 10,10′-substituted-9,9′-biacridine and signal or signals specific for said non-biacridine label to produce light;
(e) detecting or measuring said light from said reaction in (d); each analyte being differentiated and quantitated based on unique characteristics of light emitted from said labels.

30. In a ligand binding assay method for determining the presence or measuring the concentration of an unknown amount of a bio-active analyte in sample whereby such presence or concentration is determined by using label(s) and signal(s) to produce a detectable or measurable reaction product, an improvement is set out comprising using a symmetric, uniformly symmetric or asymmetric 10,10′-substituted-9,9′-biacridine of claim 4 as the label and oxidant or oxidants, chelating agent, sulfoxide, reducing sugar, alcohol., singlet oxygen, singlet oxygen generators, light, heat, energy, radiofrequency discharge, electromagnetic energy, electricity, microwave, ultrasound, chemical, singlet oxygen emitting molecules or a combination of signals capable of overcoming the energy of activation of the 10,10′-substituted-9,9′-biacridine to produce luminescence.
